SEQUENCE LISTING (29 sequences)

SEQ ID NO: 1 - DNA, 676 bp, Escherichia coli
  source 1..676: mol_type = genomic DNA
cccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacattg

SEQ ID NO: 2 - DNA, 676 bp, Escherichia coli
  source 1..676: mol_type = genomic DNA
cccgtagaaaagatcaaaagatcttcttgagatcctttttttctgcgcgtaatctgctacttgcaaacaaaaaaaccaccgctaccagcggtggtttgtttgccgaatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacattg

SEQ ID NO: 3 - DNA, 16 bp, synthetic construct
  misc_feature 1..16: note = Synthetic oligonucleotide
  source 1..16: mol_type = other DNA
ttttatctgttttttt

SEQ ID NO: 4 - DNA, 18 bp, synthetic construct
  misc_feature 1..18: note = Synthetic oligonucleotide
  source 1..18: mol_type = other DNA
ttttatctgttttttttt

SEQ ID NO: 5 - DNA, 18 bp, synthetic construct
  misc_feature 1..18: note = Synthetic oligonucleotide
  source 1..18: mol_type = other DNA
cgttttatctgttttttt

SEQ ID NO: 6 - DNA, 18 bp, synthetic construct
  misc_feature 1..18: note = Synthetic oligonucleotide
  source 1..18: mol_type = other DNA
cgttccatctgttttttt

SEQ ID NO: 7 - DNA, 18 bp, synthetic construct
  misc_feature 1..18: note = Synthetic oligonucleotide
  source 1..18: mol_type = other DNA
cgttttatctgtttgttt

SEQ ID NO: 8 - DNA, 18 bp, synthetic construct
  misc_feature 1..18: note = Synthetic oligonucleotide
  source 1..18: mol_type = other DNA
cgttttatctgttgtttt

SEQ ID NO: 9 - DNA, 582 bp, synthetic construct
  misc_feature 1..582: note = Homo sapiens EPO sequence, codon optimized, reference
  source 1..582: mol_type = other DNA
atgggtgtgcacgaatgtcctgcttggctgtggctccttctctccctgctgtccctgcctcttggactcccggtgcttggagcacccccgagactgatctgcgacagcagggtgctcgagcgctacctcctggaagccaaggaagccgaaaacatcactactggctgcgccgaacactgctccctgaacgagaacatcaccgtgccggacaccaaggtcaacttctacgcgtggaagagaatggaggtcggacagcaagccgtggaagtgtggcagggacttgcgctcctgtcggaagccgtgctgaggggacaagccctgctcgtgaacagctcacagccttgggagcccctgcagctgcatgtcgacaaggccgtgtccggactgcgctcactgaccactctgctgagggccttgggtgcccagaaagaggctatttccccaccggatgcagcctcggcagctcctctgcggaccattacggcggacacctttcggaagctgttccgcgtctacagcaatttcctccgggggaagttgaaactgtataccggcgaagcctgtcggactggcgatcgctga

SEQ ID NO: 10 - DNA, 582 bp, synthetic construct
  misc_feature 1..582: note = Homo sapiens EPO sequence, codon optimized, #1
  source 1..582: mol_type = other DNA
atgggggttcatgagtgcccagcttggctttggctcctgctcagcttgcttagtctccctttgggcctgcccgtgctgggcgcccctccacgcttgatctgtgacagcagggtcttggaacggtatttgcttgaagctaaagaagctgagaacataacaacgggatgtgctgaacattgctccttgaacgaaaacatcacagttcccgacacaaaagtcaatttttacgcatggaagcggatggaggttggccagcaagctgtggaggtctggcaagggctggctcttctcagtgaagccgtgctgcgcggacaagcactcttggtgaactccagccagccctgggagccccttcagctccatgtcgataaagcagttagcggcctccgatcattgactaccctccttagggctttgggtgcacaaaaagaggccatttcaccaccggacgcggcaagtgctgctccgttgcgaactataactgctgacaccttccggaaactttttcgggtatattccaactttctcagggggaaactcaagctctacaccggcgaggcgtgccgaactggagaccgctga

SEQ ID NO: 11 - DNA, 582 bp, synthetic construct
  misc_feature 1..582: note = Homo sapiens EPO sequence, codon optimized, #2
  source 1..582: mol_type = other DNA
atgggcgtacatgaatgcccggcatggctttggctgctgctgtccctgctgagtttgccgctgggcctccccgtcctcggcgctcccccgagactcatttgcgactctagggtcctcgaacgctatctgctggaagcaaaagaagctgagaacataactacaggatgcgctgagcactgttccttgaatgagaatatcacagtacctgacactaaggtgaatttttacgcatggaaacgcatggaagtgggtcagcaggccgtggaagtgtggcagggcctggcgctgctgtccgaggctgttcttagaggccaagccttgttggtcaattcctctcaaccctgggagcccctccagctgcatgttgataaagccgtctctggtctccggtcccttaccaccctgctcagggcacttggcgcacagaaggaagctatctcccccccagacgctgccagtgccgcccccctccggactattaccgccgatactttcaggaaactgtttcgagtctatagcaattttctccgcgggaaactgaagctgtatacaggtgaggcctgcaggacaggagatcgctga

SEQ ID NO: 12 - DNA, 18 bp, Bacteriophage SP6
  source 1..18: mol_type = genomic DNA
atttaggtgacactatag

SEQ ID NO: 13 - DNA, 23 bp, synthetic construct
  misc_feature 1..23: note = Synthetic oligonucleotide
  source 1..23: mol_type = other DNA
atttaggggacactatagaagag

SEQ ID NO: 14 - DNA, 22 bp, synthetic construct
  misc_feature 1..22: note = Synthetic oligonucleotide
  source 1..22: mol_type = other DNA
atttaggggacactatagaagg

SEQ ID NO: 15 - DNA, 23 bp, synthetic construct
  misc_feature 1..23: note = Synthetic oligonucleotide
  source 1..23: mol_type = other DNA
atttaggggacactatagaaggg

SEQ ID NO: 16 - DNA, 20 bp, synthetic construct
  misc_feature 1..20: note = Synthetic oligonucleotide
  source 1..20: mol_type = other DNA
atttaggtgacactatagaa

SEQ ID NO: 17 - DNA, 22 bp, synthetic construct
  misc_feature 1..22: note = Synthetic oligonucleotide
  source 1..22: mol_type = other DNA
atttaggtgacactatagaaga

SEQ ID NO: 18 - DNA, 23 bp, synthetic construct
  misc_feature 1..23: note = Synthetic oligonucleotide
  source 1..23: mol_type = other DNA
atttaggtgacactatagaagag

SEQ ID NO: 19 - DNA, 22 bp, synthetic construct
  misc_feature 1..22: note = Synthetic oligonucleotide
  source 1..22: mol_type = other DNA
atttaggtgacactatagaagg

SEQ ID NO: 20 - DNA, 23 bp, synthetic construct
  misc_feature 1..23: note = Synthetic oligonucleotide
  source 1..23: mol_type = other DNA
atttaggtgacactatagaaggg

SEQ ID NO: 21 - DNA, 23 bp, synthetic construct
  misc_feature 1..23: note = n is a, c, t or g
  misc_feature 22: note = n is a, c, g, or t
  source 1..23: mol_type = other DNA
atttaggtgacactatagaagng

SEQ ID NO: 22 - DNA, 24 bp, synthetic construct
  misc_feature 1..24: note = Synthetic oligonucleotide
  source 1..24: mol_type = other DNA
catacgatttaggtgacactatag

SEQ ID NO: 23 - DNA, 18 bp, Bacteriophage T7
  source 1..18: mol_type = genomic DNA
taatacgactcactatag

SEQ ID NO: 24 - DNA, 36 bp, synthetic construct
  misc_feature 1..36: note = Synthetic oligonucleotide
  source 1..36: mol_type = other DNA
ttttatctgtttttttttttttatctgttttttttt

SEQ ID NO: 25 - DNA, 54 bp, synthetic construct
  misc_feature 1..54: note = Synthetic oligonucleotide
  source 1..54: mol_type = other DNA
ttttatctgtttttttttttttatctgtttttttttttttatctgttttttttt

SEQ ID NO: 26 - protein, 874 aa, Bacteriophage SP6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 27 - DNA, 2625 bp, Bacteriophage SP6
  source 1..2625: mol_type = genomic DNA
atgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagtgcgctggatggttgatacaggtatcgaagtacctgagcaaggggagttcgaccttaacgaaatcatggattctgaatacgtatttgcctaa

SEQ ID NO: 28 - DNA, 120 bp, Escherichia coli
  source 1..120: mol_type = genomic DNA
ggcgcccttaaatattctgacaaatgctctttccctaaactccccccataaaaaaacccgccgaagcgggtttttacgttatttgcggattaacgattactcgttatcagaaccgcccag

SEQ ID NO: 29 - DNA, 301 bp, Staphylococcus aureus
  source 1..301: mol_type = genomic DNA
tattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgaca

(19) United States
(12) Patent Application Publication
(10) Pub. No.: US 2025/0075201 A1
(43) Pub. Date: Mar. 6, 2025

(54) SCREENING CODON-OPTIMIZED NUCLEOTIDE SEQUENCES

(71) Applicant: TRANSLATE BIO, INC., Waltham, MA (US)

(72) Inventors: Minnie ZACHARIA, Waltham, MA (US); Nicholas DREISBACH, Waltham, MA (US); Anusha DIAS, Waltham, MA (US)

(21) Appl. No.: 18/580,463

(22) PCT Filed: Aug. 15, 2022
(86) PCT No.: PCT/US2022/074975

§ 371 Date: Jan. 18, 2024

Related U.S. Application Data
(60) Provisional application No. 63/233,658, filed on Aug. 16, 2021.

(51) Int. Cl. C12N 15/10; C12N 15/66; G16B 35/10

(52) U.S. Cl. CPC C12N 15/1089; C12N 15/1013; C12N 15/66; G16B 35/10

(57) ABSTRACT

The present invention relates to methods for screening protein-coding nucleotide sequences generated by a codon optimization algorithm to identify those sequences that generate a full-length mRNA transcript, and optionally, high protein expression. In particular, the present invention relates to screening methods wherein a plurality of protein-coding nucleotide sequences is provided as two or more DNA fragments which are assembled via homologous ends into plasmids that comprise the nucleotide sequences of interest flanked by a 5′ untranslated region (5′ UTR) and a 3′ untranslated region (3′ UTR) and operationally linked to an RNA polymerase promoter.

### CROSS-REFERENCE TO RELATED APPLICATIONS

This application claims benefit of, and priority to U.S. Provisional Patent Application Ser. No. 63/233,658 filed on Aug. 16, 2021, the contents of which are incorporated herein in its entirety.

### FIELD OF THE INVENTION

The present invention relates to methods for screening protein-coding nucleotide sequences generated by a codon optimization algorithm to identify those sequences that generate a full-length mRNA transcript, and optionally, high protein expression. In particular, the present invention relates to screening methods wherein a plurality of protein-coding nucleotide sequences is provided as two or more DNA fragments which are assembled via homologous ends into plasmids that comprise the nucleotide sequences of interest flanked by a 5′ untranslated region (5′ UTR) and a 3′ untranslated region (3′ UTR) and operationally linked to an RNA polymerase promoter.

### SEQUENCE LISTING

The present specification makes reference to a Sequence Listing submitted electronically which is named MRT-2251WO1_ST.26XML. The file was generated on Jul. 20, 2022, and is 41,917 bytes in size. The entire contents of the sequence are herein incorporated by reference.

### BACKGROUND OF THE INVENTION

mRNA therapy is increasingly important for treating various diseases, especially those caused by dysfunction of proteins or genes. Genetic mutations in the DNA sequence of an organism can lead to aberrant gene expression, resulting in defects in protein production or function. For example, mutations in an underlying DNA sequence can lead to insufficient expression or over-expression of a protein, or production of dysfunctional proteins. Restoration of normal or healthy levels of the protein can be achieved through mRNA therapy, which is widely applicable to a range of diseases caused by gene or protein dysfunction.

In mRNA therapy, mRNA encoding a functional protein that can replace a defective or missing protein is delivered to a target cell or tissue. Administration of an mRNA encoding a therapeutic protein efficacious in treating or preventing a disease or disorder can also provide a cost-effective alternative to therapy with a recombinantly produced peptide, polypeptide or protein. mRNA therapy can restore the normal levels of an endogenous protein or provide an exogenous therapeutic protein without permanently altering the genome sequence or entering the nucleus of the cell. mRNA therapy takes advantage of the cells' own protein production and processing machinery to treat diseases or disorders, is flexible to tailored dosing and formulation, and is broadly applicable to any disease or condition caused by an underlying gene or protein defect or treatable through the provision of an exogenous protein.

Vaccines based on mRNA therapy enable expression of complex antigens in their natural conformation and with their natural post-translational modifications in an immunized subject. Unlike traditional technologies, the manufacture of mRNA vaccines does not require complex and costly bacterial fermentation, tissue culture, or purification processes.

The efficacy and therapeutic benefits of mRNA therapy can be significantly impacted by expression levels of an mRNA-encoded protein. To promote higher expression of the mRNA-encoded protein, optimization of the composition and order of codons within a protein-coding nucleotide sequence (“codon optimization”) is often performed. However, codon optimization may not remove, and in some instances may introduce, cryptic transcription termination sites or sequence motifs that interfere with the efficient transcription of the nucleotide sequence, e.g., by leading to premature termination during in vitro transcription. Moreover, other parameters such as guanine-cytosine (GC) content may impact efficient expression of a nucleotide sequence.

Sequence optimization algorithms typically generate several candidate “optimized” protein-coding nucleotide sequences. These candidate sequences commonly are cloned into template plasmids that provide the necessary elements (e.g., an RNA polymerase promoter) to test their performance in an in vitro synthesis reaction, in particular to confirm that the optimized nucleotide sequence is transcribed into a full-length mRNA transcript. Often it is also necessary to confirm empirically that the resulting mRNA transcripts have the ability to direct high protein expression.

Current screening methods for codon-optimized protein-coding nucleotide sequences are resource intensive and difficult to perform at scale, hampering efforts to identify optimized sequences that are suitable for the efficient commercial production of full-length mRNA transcripts at an early development stage. Accordingly, a need exists for improved methods that can easily be performed at scale to screen large numbers of optimized protein-coding nucleotide sequences.

### SUMMARY OF THE INVENTION

The present invention addresses the need for methods that can easily be performed at scale to screen large numbers of optimized protein-coding nucleotide sequences. In particular, the methods of the invention build on advances in the efficient chemical synthesis of short DNA fragments and long DNA fragments, and molecular biology techniques that allow their rapid and accurate assembly via one or more sets of homologous ends.

For example, a nucleotide sequence encoding a protein that was generated by a sequence optimization algorithm (e.g., a codon optimization algorithm) can be divided computationally into DNA fragments with overlapping homologous ends which allows their easy re-assembly by highly efficient molecular biology techniques such as Gibson assembly. The DNA fragments can be prepared by chemical synthesis with an accuracy of less than 1 error in 5,000 bases, such that quantities (typically at 250-1000 ng) of error-free DNA fragments of 4,000 base pairs (bp) can be provided by commercial suppliers within a few days of ordering at low cost. Including delivery times, assembly of the DNA fragments via homologous ends and insertion of the resulting nucleotide sequence into a vector backbone using commercially available reagents yields within 4-5 days a plasmid which can be used as the template for the in vitro synthesis of mRNA transcripts (the assembly itself can be done in 1-2 days). Shorter DNA fragments (less than 1,500 bp in length, e.g., about 1,000 bp in length) may assemble more efficiently using techniques such as Gibson assembly.

Either the plasmid backbone or the DNA fragments may provide a 5′ untranslated region (5′ UTR), a 3′ untranslated region (3′ UTR), and an RNA polymerase promoter. 5′ and 3′ UTRs are typically located on the respective ends of the protein coding nucleotide sequence in a mature mRNA. The RNA polymerase promoter enables transcription of the nucleotide sequence when the assembled plasmid is added to an in vitro transcription reaction mixture.

For efficient assembly of a plasmid, it may be advantageous to include the 5′ UTR and the 3′ UTR sequences in the DNA fragments. Doing so may prevent the plasmid backbone from circularizing without an insert encoding the nucleotide sequence of interest. The plasmid backbone can be provided in the form of multiple vector fragments, which may make the assembly of a plasmid containing the nucleotide sequence of interest more efficient.

By utilizing an optimized workflow, the inventors have found that they can screen protein-coding nucleotide sequences generated by a sequence optimization algorithm within a couple of weeks of their original design, making it possible to provide in less than a month a template plasmid comprising an optimized nucleotide sequence which is suitable for the production of full-length mRNA transcripts. mRNA transcripts produced from such a plasmid typically have a high in vivo potency.

In particular, the invention relates, among other things, to a screening method that comprises the following steps:
a. providing a plurality of nucleotide sequences encoding a protein generated by a sequence optimization algorithm;
b. for each nucleotide sequence, providing two or more DNA fragments with a first set of homologous ends and a second set of homologous ends, wherein said two or more DNA fragments, when assembled via the first set of homologous ends, yield an insert with the second set of homologous ends and comprising the nucleotide sequence;
c. providing two or more vector fragments with the second set of homologous ends and a third set of homologous ends, wherein said two or more vector fragments, when assembled via the third set of homologous ends, yield a vector backbone with the second set of homologous ends;
d. for each nucleotide sequence, assembling the two or more DNA fragments and the two or more vector fragments via the first, second and third sets of homologous ends, wherein assembly of the insert and the vector backbone via the second set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

The invention also relates to a screening method that comprises the following steps:
a. providing a plurality of nucleotide sequences encoding a protein generated by a sequence optimization algorithm;
b. for each nucleotide sequence, providing two or more DNA fragments with a first set of homologous ends and a second set of homologous ends, wherein said two or more DNA fragments, when assembled via the first set of homologous ends, yield an insert with the second set of homologous ends and comprising the nucleotide sequence;
c. providing a vector backbone with the second set of homologous ends;
d. for each nucleotide sequence, assembling the two or more DNA fragments and the vector backbone via the first and second sets of homologous ends, wherein assembly of the insert and the vector backbone via the second set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

In some instances, the nucleotide sequence of interest is short enough (e.g., less than 4000 bp, e.g., less than 1,500 bp) to be effectively prepared by chemical synthesis and inserted into a vector backbone that has optionally been divided into vector fragments for efficient assembly. Accordingly, the present invention also provides a screening method that comprises the following steps:
a. providing a plurality of nucleotide sequences encoding a protein generated by a sequence optimization algorithm;
b. for each nucleotide sequence, providing an insert with a first set of homologous ends and comprising the nucleotide sequence;
c. providing two or more vector fragments with the first set of homologous ends and a second set of homologous ends, wherein said two or more vector fragments, when assembled via the second set of homologous ends, yield a vector backbone with the first set of homologous ends;
d. for each nucleotide sequence, assembling the insert and the two or more vector fragments via the first and second sets of homologous ends, wherein assembly of the insert and the vector backbone via the first set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

In some embodiments, the two or more vector fragments in step (c) in the screening methods described in the preceding paragraphs may be provided by means of a DNA polymerase and a plurality of primer pairs encoding the second and third sets of homologous ends or the first and second sets of homologous ends, as applicable.

In some instances, the nucleotide sequence of interest and the vector backbone in which it is to be inserted are both short enough (e.g., less than 4000 bp, e.g., less than 1,500 bp) so that is it not necessary to provide the nucleotide as two or more DNA fragments and the vector backbone as two or more vector fragments. Alternatively, due to advances in the ability to accurately, reliably and rapidly synthesize high-quality long DNA fragments at scale, the insert comprising the nucleotide sequence of interest (and/or the vector backbone) may be provided without the need to generate two or more DNA fragments. Accordingly, the invention also relates to a screening method that comprises the following steps:
a. providing a plurality of nucleotide sequences encoding a protein generated by a sequence optimization algorithm;
b. for each nucleotide sequence, providing an insert comprising the nucleotide sequence and a set of homologous ends;
c. providing a vector backbone comprising the set of homologous ends;
d. for each nucleotide sequence, assembling the insert and the vector backbone via the set of homologous ends, wherein the assembly yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

A screening method in accordance with the invention may further comprise the following additional steps in order to ascertain that a selected nucleotide sequence is efficiently expressed:
g. for each nucleotide sequence selected in step (f) of any one of the methods described in the preceding paragraphs, transfecting a cell with the full-length mRNA transcript;
h. for each cell transfected in step (g), determining the amount of the encoded protein expressed from the full-length mRNA transcript; and
i. selecting the nucleotide sequence, whose full-length mRNA transcript yields the largest amount of the encoded protein.

A sequence optimization algorithm (e.g., a codon optimization algorithm) for generating a plurality of nucleotide sequences encoding a protein of interest which are then the subject of a screening method of the invention may comprise the following steps:
(i) receiving an amino acid sequence encoding the protein;
(ii) receiving a first codon usage table, wherein the first codon usage table comprises a list of amino acids, wherein each amino acid in the table is associated with at least one codon and each codon is associated with a usage frequency;
(iii) removing from the codon usage table any codons associated with a usage frequency which is less than a threshold frequency;
(iv) generating a normalized codon usage table by normalizing the usage frequencies of the codons not removed in step (iii);
(v) generating a nucleotide sequence encoding the amino acid sequence by selecting a codon for each amino acid in the amino acid sequence based on the usage frequency of the one or more codons associated with the amino acid in the normalized codon usage table; and
(vi) repeating step (v) to generate the plurality of nucleotide sequences.

Such a codon optimization algorithm may further comprise the following additional steps:
(vii) determining the codon adaptation index of each of the nucleotide sequences, wherein the codon adaptation index of a sequence is a measure of codon usage bias and can be a value of 0 to 1; and
(viii) removing any nucleotide sequence if its codon adaptation index is less than or equal to a predetermined codon adaptation index threshold.

In certain embodiments, the codon adaptation index threshold may be 0.7, or 0.75, or 0.85, or 0.9, or, in particular, 0.8.

Alternatively or in addition, a sequence optimization algorithm for generating a plurality of nucleotide sequences encoding a protein of interest which are then the subject of a screening method of the invention may comprise the following steps:
i. determining whether any one of the nucleotide sequences contains a termination signal; and
ii. removing any nucleotide sequence if the nucleotide sequence contains one or more termination signals.

The one or more termination signals may have the nucleic acid sequence 5′-X₁ATCTX₂TX₃-3′, wherein X₁, X₂ and X₃ are independently selected from A, C, T or G. For example, the one or more termination signal may have one or more of the following nucleotide sequences:

TATCTGTT;
and/or
TTTTTT;
and/or
AAGCTT;
and/or
GAAGAGC;
and/or
TCTAGA.

Each of the nucleotide sequences provided in step (a) in a screening method of the invention may be processed by a fragmentation algorithm, wherein the fragmentation algorithm divides each nucleotide sequence into two or more nucleic acid fragments and adds the homologous ends required for the assembling of the plasmid performed in step (d) of the screening method.

Each of the two or more DNA fragments or the insert, as applicable, may be provided by a chemical synthesis process. In some embodiments, the two or more DNA fragments or the insert is about 1000 base pairs long. In other embodiments, the two or more DNA fragments or the insert is at least 1000 base pairs long. For example, each of the two or more DNA fragments or the insert may be 1000 base pairs to 4000 base pairs long. In some embodiments, each of the two or more DNA fragments or the insert are/is 1000 base pairs to 7000 base pairs long. In other embodiments, each of the two or more DNA fragments or the insert are/is 1000 base pairs to 20,000 base pairs long.

In certain embodiments, the chemical synthesis process that is used to provide the two or more DNA fragments or the insert has a median error rate of less than or equal to 1 error per 5000 base pairs. In one particular embodiment, the chemical synthesis process has a median error rate of less than or equal to 1 error per 10,000 base pairs. In another particular embodiment, the chemical synthesis process has a median error rate of less than or equal to 1 error per 50,000 base pairs.

Chemical synthesis processes with a very low error rate (e.g., less than or equal to 1 error per 30,000 base pairs, particularly less than or equal to 1 error per 50,000 base pairs) are particularly suitable for generating large inserts of up to 7000 base pairs, or, in some instances, up to 20,000 base pairs. Such chemical synthesis processes may be employed together with a method as described herein that uses a vector backbone and a single insert, rather than two or more DNA fragments, to rapidly assemble plasmids for the screening of nucleotide sequences of interest.

The homologous ends, which are required for assembly of the insert comprising the nucleotide sequence of interest, may be 15 base pairs to 30 base pairs long.

In some embodiments, the assembling in step (d) of a screening method of the invention is performed in the presence of a DNA polymerase and/or an exonuclease, and optionally a ligase. In a specific embodiment, the assembling in step (d) of a screening method of the invention is performed in the presence of a 5′ exonuclease, a DNA polymerase and a ligase.

In some embodiments, the vector backbone comprises a negative selection marker gene and/or a positive selection marker gene. In some embodiments, the vector backbone comprises an origin of replication having the nucleotide sequence of SEQ ID NO: 1. In some embodiments, the vector backbone comprises an origin of replication with a single base substitution having the nucleotide sequence of SEQ ID NO: 2.

In some embodiments, the plasmid comprises a bacterial terminator located in the vector backbone upstream of the RNA polymerase promoter. In some embodiments, the bacterial terminator is an Escherichia coli ropC terminator. In other embodiments, the bacterial terminator is a Staphylococcus aureus bla terminator.

In certain embodiments, it may be advantageous to use a circular plasmid that comprises two or more termination signals arranged sequentially and positioned at the 3′ end of the 3′UTR. The presence of the two more termination signals at the 3′ end may avoid the need for linearization of the plasmid prior to in vitro transcription of the nucleotide sequence in step (e) of a screening method of the invention. Accordingly, in some embodiments, the vector backbone comprises two or more termination signals arranged sequentially and positioned at the 3′ end of the 3′UTR in the plasmid. In a specific embodiment, the plasmid comprises three termination signals. In a typical embodiment, each termination signal is separated by 10 base pairs or fewer. For example, each termination signal may be separated by 5 to 10 base pairs.

Each termination signal may comprise the following nucleic acid sequence 5′-X₁ATCTX₂TX₃-3′, wherein X₁, X₂ and X₃ are independently selected from A, C, T or G. In particular embodiments, each termination signal comprises the nucleic acid sequence 5′-X₁ATCTGTT-3′. In one specific embodiment, X₁ is T. In another specific embodiment, X₁ is C. In particular embodiments, the termination signal is selected from

| (SEQ ID NO: 3) | |
|---|---|
| | 5′-TTTTATCTGTTTTTTT-3′, |
| (SEQ ID NO: 4) | |
| | 5′-TTTTATCTGTTTTTTTTT-3′, |
| (SEQ ID NO: 5) | |
| | 5′-CGTITTATCTGTTTTTTT-3′, |
| (SEQ ID NO: 6) | |
| | 5′-CGTTCCATCTGTTTTTTT-3′, |
| (SEQ ID NO: 7) | |
| | 5′-CGTTTTATCTGTTTGTTT-3′, |
| | or |
| (SEQ ID NO: 8) | |
| | 5′-CGTTTTATCTGTTGTTTT-3′. |

In some embodiments, the plasmid is linearized before step (e) of a screening method of the invention.

In some embodiments, the RNA polymerase promoter is an SP6 polymerase promoter.

In some embodiments, a screening method of the invention further comprises one or more purification steps prior to performing step (e). In a specific embodiment, the one or more purification steps comprises purifying the plasmid to remove one or more enzyme(s) used for assembly in step (d). In another specific embodiment, the one or more purification steps comprises extracting the plasmid from Escherichia coli cells.

In a specific embodiment, the purification comprises precipitating the plasmid by adding (i) a chaotropic salt, and (ii) an alcohol and/or an amphiphilic polymer. In some embodiments, the chaotropic salt is at a final concentration of 0.1-4 M, for example, about 125 mM, about 250 mM, about 375 mM, about 500 mM, about 625 mM, about 750 mM, about 1.3 M, about 1.9 M or about 2.5 M. In another specific embodiment, the chaotropic salt is guanidinium salt, e.g., guanidinium thiocyanate (GSCN). In some embodiments, the amphiphilic polymer is selected from pluronics, polyvinyl pyrrolidone, polyvinyl alcohol, polyethylene glycol (PEG), triethylene glycol monomethyl ether (MTEG), or combinations thereof. In a specific embodiment, the amphiphilic polymer is MTEG. In some embodiments, the alcohol is isopropanol or ethanol.

In some embodiments, the in vitro transcription reaction mixture in step (e) of a screening method of the invention comprises the plasmid at a concentration of 0.05 mg/ml or greater (e.g., 0.07 mg/ml or greater). In some embodiments, the in vitro transcription reaction mixture comprises an RNA polymerase at a concentration of 0.1 mg/ml or greater.

In a particular embodiment, steps (b)-(f) of a screening method of the invention are performed in 96-well plates.

In some embodiments, steps (g)-(i) of a screening method of the invention are performed in 96-well plates. In some embodiments, the cell transfected in step (h) is a mammalian cell. In a specific embodiment, the mammalian cell is a human cell.

Also provided is a high-throughput method for purifying a plurality of DNA constructs, wherein the method comprises performing for each DNA construct the following steps in parallel:
a. providing an impure preparation comprising the DNA construct in a first receptacle;
b. adding (i) a chaotropic salt, (ii) an alcohol and/or an amphiphilic polymer, and optionally (iii) a buffered solution to the impure preparation under conditions that result in the formation of a precipitate comprising the DNA construct;
c. adding a DNA-binding magnetic particle to bind the precipitate formed in step (b) to the magnetic particle;
d. transferring the magnetic particle with the bound precipitate from the first receptacle to a second receptacle comprising a first wash solution;
e. optionally transferring the magnetic particle with the bound precipitate from the second receptacle to a third receptacle comprising a second wash solution;
f. transferring the magnetic particle with the bound precipitate from the wash solution to a fourth receptacle comprising an elution medium; and
g. solubilizing the precipitate in the elution medium to release the purified DNA construct.

In some embodiments, each of the first, second, third and fourth receptacles is a well in a first, second, third and fourth multi-well plate, respectively.

In some embodiments, each multi-well plate is a 96-well plate.

In some embodiments, each step in the high-throughput purification method is performed by an automated liquid handling system.

In some embodiments, the magnetic particle is a silica-coated bead with a metallic core. In specific embodiments, the metallic core comprises iron, nickel or cobalt.

In some embodiments, the chaotropic salt is at a final concentration of 0.1-4 M to form the precipitate in step (b) of the high-throughput purification method. In specific embodiments, the chaotropic salt is at a final concentration of 1.5 M-2.7 M. In some embodiments, the chaotropic salt is a guanidinium salt, e.g., guanidinium thiocyanate (GSCN).

In some embodiments, the amphiphilic polymer is selected from pluronics, polyvinyl pyrrolidone, polyvinyl alcohol, polyethylene glycol (PEG), triethylene glycol monomethyl ether (MTEG), or combinations thereof. In a specific embodiment, the amphiphilic polymer is MTEG. In some embodiments, the amphiphilic polymer is present at about 30% (v/v) to about 70% (v/v) final concentration to form the precipitate in step (a) of the high-throughput purification method.

In some embodiments, the alcohol is isopropanol or ethanol. In some embodiments, the isopropanol or ethanol is present at about 30% (v/v) to about 70% (v/v) final concentration to form the precipitate in step (a) of the high-throughput purification method.

In some embodiments, the buffered solution has a pH of about 5 to about 6. In some embodiments, the buffered solution comprises potassium acetate. In some embodiments, an amount of the buffered solution is provided in step (b) of the high-throughput purification method to obtain a final potassium acetate concentration of about 1 M to about 2 M.

In some embodiments, the first wash solution is 100% isopropanol or 80% (v/v) ethanol. In some embodiments, the second wash solution is 100% isopropanol or 80% (v/v) ethanol. In specific embodiments, the first wash solution is 100% isopropanol and the second wash solution is 80% (v/v) ethanol. In some embodiments, the first wash solution and the second wash solution are 80% (v/v) ethanol. In particular embodiments, the first wash solution and the second wash solution are 100% isopropanol.

In some embodiments, the elution medium is sterile water. In some embodiments, the elution medium is heated to a temperature of 30° C. to 50° C. to enhance solubilization of the precipitate.

In some embodiments, the impure preparation is a cell lysate. In particular embodiments, the cell is a bacterial cell. In some embodiments, the lysate is an alkaline solution. In some embodiments, the lysate comprises a detergent.

In some embodiments, the high-throughput purification method further comprises determining the concentration of the purified DNA construct obtained in step (g).

In some embodiments, the high-throughput purification method further comprises lyophilizing the purified DNA construct obtained in step (g).

In some embodiments, each DNA construct in the plurality of DNA constructs is a plasmid. In particular embodiments, each plasmid comprises a nucleotide sequence, said nucleotide sequence being flanked by a 5′ untranslated region (5′ UTR) and a 3′ untranslated region (3′ UTR) and operationally linked to an RNA polymerase promoter. In a specific embodiment, each preparation of purified plasmid obtained in step (g) of the high-throughput purification method comprises the chaotropic salt at a concentration that does not interfere with in vitro transcription of the nucleotide sequence. In another specific embodiment, the nucleotide sequence was generated by a codon optimization algorithm.

### BRIEF DESCRIPTION OF THE DRAWINGS

Embodiments of the invention will be described, by way of example, with reference to the following drawings, in which:

FIG. 1 illustrates the overall scheme of the screening methods according to the invention.

FIG. 2 illustrates a codon optimization method according to an embodiment of the present invention.

FIG. 3 illustrates a particular embodiment of the invention in which a motif screen filter, guanine-cytosine (GC) content analysis filter, and codon adaptation index (CAI) analysis filter have been applied, in that order, to the list of optimized nucleotide sequences. In a particular embodiment, the list of optimized nucleotide sequences for filtering has been generated according to a method as shown in FIG. 1.

FIG. 4 illustrates an example repository of nucleotide sequence motifs which includes a termination signal, suitable for use in removing nucleotide sequences containing one more termination signal.

FIG. 5 illustrates an example analysis of the guanine-cytosine (GC) content of non-optimized and optimized nucleotide sequences, wherein the guanine-cytosine (GC) content of portions of the nucleotide sequence encoding EPO is determined for adjacent non-overlapping portions 30 nucleotides in length.

FIG. 6A schematically illustrates a vector backbone suitable for use with the screening methods of the invention. The circular plasmid shown in FIG. 6A is linearized to accept an insert encoding a nucleotide sequence of interest between a 5′ UTR and 3′ UTR via suitable homologous ends. FIG. 6B shows part of a circular plasmid that includes additional components (e.g., DNA inserts). The plasmid contains a nucleotide sequence of interest which was inserted between a 5′ UTR and 3′ UTR and is operationally linked to an SP6 RNA polymerase promoter. The 5′ UTR and 3′ UTR sequences are flanked by first and second polylinkers. In addition, a bacterial terminator sequence (a Staphylococcus aureus bla terminator) is located immediately upstream of the SP6 RNA polymerase promoter to prevent transcription of the nucleotide sequence of interest by bacterial RNA polymerases during plasmid amplification in a bacterial cell.

FIG. 7 schematically illustrates linearization and/or fragmentation of a vector backbone and assembly of a plasmid comprising the vector backbone and an insert assembled from DNA fragments encoding a nucleotide sequence of interest. Assembly of the vector fragments and/or DNA fragments is done via homologous ends. Homologous ends are generated by dividing the nucleotide sequence of interest and the vector backbone into overlapping DNA fragments and overlapping vector fragments, respectively, as shown schematically in FIG. 7. The DNA fragments and vector fragments include an additional set of homologous ends (shown in light grey) for assembly into a plasmid. The DNA fragments are assembled into an insert via overlapping regions (homologous ends), and the vector fragments are assembled into a vector backbone via overlapping regions (homologous ends). The additional set of homologous ends serves to assemble the insert and the vector backbone into a circular plasmid as shown.

FIG. 8A and FIG. 8B show representative agarose gels with the products of PCR reactions performed with primers flanking an insert to verify the correct assembly of DNA fragments/inserts and a vector backbone. FIG. 8A shows the product of a PCR reaction performed directly on the assembly reaction product. FIG. 8B shows the product of a PCR reaction performed on an E. coli colony transformed with the assembly reaction product. The presence of an amplified insert band for all of the test reactions demonstrates that the assembly reactions were successful with a range of vector backbone and insert fragment numbers and sizes. Unreacted vector backbone was observed when performing PCR directly on the assembly reaction product (see FIG. 8A).

FIG. 9A shows the result of gel electrophoresis analysis of 6 test plasmids purified according to the QIAGEN® standard protocol. FIG. 9B shows the results of a gel electrophoresis analysis for 7 plasmids precipitated from cleared cell lysates by adding guanidinium thiocyanate (GSCN) and triethylene glycol monomethyl ether (MTEG) and washed with 100% isopropanol. Both uncut (UC) and linearized (L) plasmid DNA was run on the agarose gel shown in FIG. 9B. 1 μg of each of the purified plasmid preparations was added to the wells of the gel shown in FIG. 9A, and 0.5 μg was loaded into the wells of the gel shown in FIG. 9B. As can be seen from FIG. 9B, the yield was much improved when GSCN/MTEG precipitation was used for purification.

FIG. 10 illustrates a typical analysis of in vitro transcription (IVT) reactions by capillary electrophoresis. These results are shown as a digitally generated gel based on the fluorescence signal obtained for each sample. Full-length mRNA was transcribed from each of the plasmids comprising the nucleotide sequence that had been generated with a codon optimization algorithm as described herein.

FIG. 11 illustrates that optimizing the concentration of plasmid and RNA polymerase in the in vitro transcription reaction mixture improves the mRNA transcript yield. FIG. 11 shows the total RNA yield at varying concentrations of linearized plasmid. Using an in vitro transcription reaction mixture with a total amount of 6 μg of linearized plasmid DNA at a final concentration of about 0.07 mg/ml was found to be particularly suitable for achieving a high mRNA transcript yield. Using less than 6 μg template DNA reduced mRNA yield. Using more than 6 μg template DNA had little to no benefit to yield.

FIG. 12A and FIG. 12B illustrate that adjusting the amount of poly(A) polymerase by assigning mRNA transcripts to bins based on their length results in a more uniform tail length. In the experiment shown in FIG. 12A, the same amount of poly(A) polymerase was used to tail mRNA transcripts of different lengths (encoding nucleotide sequences A-F; see Table 4). In the experiment shown in FIG. 12B, the amount of poly(A) polymerase in the tailing reaction was assigned based on the length of nucleotide sequences A-F. The reaction mixtures for mRNA transcripts of nucleotide sequences with a length of 0 to 1 kilobases were assigned a specified amount of poly(A) polymerase in accordance with the manufacturer's instructions (“bin 1”). The reaction mixtures for mRNA transcripts of nucleotide sequences with a length of 1 to 2 kilobases were assigned about half of the specified amount (“bin 2”), reaction mixtures for mRNA transcripts of nucleotide sequences with a length of 2 to 4 kilobases (“bin 3”) were assigned about one quarter of the specified amount, and reaction mixtures for mRNA transcripts of nucleotide sequences with a length of 4 to 8 kilobases (“bin 4”) were assigned about one eighth of the specified amount. In both experiments, the total amount of mRNA in each tailing reaction was the same. Binned tailing resulted in a more uniform poly(A) tail of about 60 nucleotides to about 300 nucleotides in length for all tested mRNA transcripts (see FIG. 12B). Most mRNA transcripts had a length of about 150-200 nucleotides. In contrast, “one size fits all” tailing reactions resulted in tail lengths ranging from less than 100 nucleotides to over 800 nucleotides (see FIG. 12A).

FIG. 13A and FIG. 13B illustrate the use of magnetic particles for the purification of plasmid DNA from an impure plasmid preparation. FIG. 13A shows the yields achieved when liquids and magnetic particles were transferred manually into and between 96-well plates. FIG. 13B illustrates how an automatic liquid handling system more than halved processing time and dramatically increased the yield of recovered plasmid DNA.

### DETAILED DESCRIPTION OF THE INVENTION

### Definitions

In order for the present invention to be more readily understood, certain terms are first defined below. Additional definitions for the following terms and other terms are set forth throughout the Specification.

As used in this Specification and the appended claims, the singular forms “a”, “an” and “the” include plural referents unless the context clearly dictates otherwise.

Unless specifically stated or obvious from context, as used herein, the term “or” is understood to be inclusive and covers both “or” and “and”.

The terms “for example”, “for instance”, “e.g.” and “i.e.” as used herein, are used interchangeably and merely by way of example, without limitation intended, and should not be construed as referring only those items explicitly enumerated in the specification.

The terms “or more”, “at least”, “more than”, and the like, e.g., “at least one” are understood to include, but are not limited, to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, and the like, are understood to include, but are not limited to, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Unless specifically stated or evident from context, as used herein, the term “about” is understood to be within a range of normal tolerance in the art, for example, within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflect normal fluctuations that can be appreciated by a skilled artisan.

As used herein, the terms “abortive transcript” or “pre-aborted transcript” or the like are any transcript that is shorter than a full-length mRNA molecule encoded by the DNA template that results from the premature release of RNA polymerase from the template DNA in a sequence-independent manner. In some embodiments, an abortive transcript may be less than 90% of the length of the full-length mRNA molecule that is transcribed from the target DNA molecule, e.g., less than 80%, 70%, 60%, 50%, 40%, 30%, 20%, 10%, 5%, 1% of the length of the full-length mRNA molecule.

As used herein, the terms “codon” and “codons” refer to a sequence of three nucleotides which together form a unit of the genetic code. Each codon corresponds to a specific amino acid or stop signal in the process of translation or protein synthesis. The genetic code is degenerate, and more than one codon can encode a specific amino acid residue. For example, codons can comprise DNA or RNA nucleotides.

As used herein, “full-length mRNA” is as characterized when using a specific assay, e.g., gel electrophoresis and detection using UV and UV absorption spectroscopy with separation by capillary electrophoresis. In a typical situation, at least 80% (e.g., 85%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, or 99.9%) of the mRNA molecule that is transcribed from the target DNA is full-length.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “messenger RNA (mRNA)” refers to a polyribonucleotide that encodes at least one polypeptide. mRNA, as used herein, encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

As used herein, the term “nucleic acid”, in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid”, “DNA”, “RNA”, and/or similar terms include nucleic acid analogs, i.e., analogs having other than a phosphodiester backbone. A nucleic acid sequence is presented in the 5′ to 3′ direction unless otherwise indicated.

As used herein, the term “nucleotide sequence”, in its broadest sense, refers to the order of nucleobases within a nucleic acid. In some embodiments, “nucleotide sequence” refers to the order of individual nucleobases within a gene. In some embodiments, “nucleotide sequence” refers to the order of individual nucleobases within a protein-coding gene. In some embodiments, “nucleotide sequence” refers to the order of individual nucleobases within single and/or double stranded DNA and/or cDNA. In some embodiments, “nucleotide sequence” refers to the order of individual nucleobases within RNA. In some embodiments, “nucleotide sequence” refers to the order of individual nucleobases within mRNA. In a particular embodiment, “nucleotide sequence” refers to the order of individual nucleobases within the protein-coding sequence of RNA or DNA. A nucleotide sequence is normally presented in the 5′ to 3′ direction unless otherwise indicated.

As used herein, the term “premature termination” refers to the termination of transcription before the full length of the DNA template has been transcribed. As used herein, premature termination can be caused by the presence of a nucleotide sequence motif (also referred to herein simply as “motif”), e.g., a termination signal, within the DNA template and results in mRNA transcripts that are shorter than the full-length mRNA (“prematurely terminated transcripts” or “truncated mRNA transcripts”). Examples of a termination signal include the E. coli rmB terminator t1 signal (consensus sequence: ATCTGTh) and variants thereof, as described herein.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore, the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### Providing Nucleotide Sequences of Interest for Screening

Step (a) of a screening method of the invention provides a plurality of nucleotide sequences encoding a protein generated by a sequence optimization algorithm, e.g., a codon optimization algorithm.

As used herein, the terms “codon optimization” and “codon-optimized” refer to modifications of the codon composition of a naturally occurring or wild-type nucleic acid encoding a peptide, polypeptide or protein that do not alter its amino acid sequence, thereby improving protein expression of said nucleic acid. Further computational analysis steps (e.g., one or more motif screens, and/or a GC content analysis) can be performed to select a subset of “optimized” nucleotide sequences.

Accordingly, in the context of the present invention, the terms “sequence optimization” or “codon optimization” may also refer to a process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

### Generating a Plurality of Nucleotide Sequences

In some embodiments, a codon-optimized nucleotide sequence is generated by selecting a codon, for each amino acid in an amino acid sequence of a protein of interest, based on the usage frequency of the one or more codons associated with the amino acid in a normalized codon usage table. The optimized nucleotide sequence is generated by arranging the selected codons in the order in which their associated amino acid appears in the amino acid sequence.

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome. These calculations can be performed on nucleotide sequences found, for example, in publicly accessible repositories and/or databases.

A codon usage table specifies the usage frequency of each codon in a given organism. Each amino acid in the table is associated with at least one codon, and each codon is associated with a usage frequency. Codon usage tables are stored in publicly available databases, such as the Codon Usage Database (Nakamura et al. (2000) Nucleic Acids Research 28(1), 292; available online at www.kazusa.or.jp/codon/), and the High-performance Integrated Virtual Environment-Codon Usage Tables (HIVE-CUTs) database (Athey et al., (2017), BMC Bioinformatics 18(1), 391; available online at hive.biochemistry.gwu.edu/review/codon).

A codon optimization algorithm for use with the screening method of the invention may receive an amino acid sequence of a protein of interest and a first codon usage table which reflects the frequency of each codon in a given organism (e.g., a human). The process then removes codons from the first codon usage table if they are associated with a codon usage frequency which is less than a threshold frequency (e.g., 10%). The codon usage frequencies of the codons not removed in the first step are normalized to generate a normalized codon usage table.

Normalizing the codon usage table involves re-distributing the usage frequency value for each removed codon; the usage frequency for a certain removed codon is added to the usage frequencies of the other codons with which the removed codon shares an amino acid. In this example, the re-distribution is proportional to the magnitude of the usage frequencies of the codons not removed from the table. The codon optimization algorithm uses the normalized codon usage table to generate a plurality of codon-optimized nucleotide sequences. Each of the optimized nucleotide sequences encode the amino acid sequence of the protein of interest.

The generation of a codon-optimized nucleotide sequence is based on a probabilistic selection of codons. The list may include any number of duplicate codon-optimized nucleotide sequences, i.e., identical optimized nucleotide sequences, because the generation of a codon-optimized nucleotide sequence is based on a probabilistic selection of replacement codons. Identical optimized sequences are removed when generating the plurality of nucleotide sequences for use in the screening method of the invention.

### Filtering the Plurality of Codon-Optimized Nucleotide Sequences

The number of codon-optimized nucleotide sequences may depend at least upon the length and content of the amino acid sequence of the protein of interest, the value of the threshold codon usage frequency, the content of the first codon usage table, and the number of times the codon optimization algorithm is run, i.e., the number of times an optimized nucleotide sequence is generated. For example, a plurality of nucleotide sequences may comprise 10,000 or more codon-optimized nucleotide sequences.

It may, therefore, be desirable to reduce the number of codon-optimized nucleotide sequences in the plurality of nucleotide sequences before, e.g., synthesis. This may advantageously reduce the time it takes to synthesize every sequence in the list and the resources necessary to do so.

Accordingly, in a typical embodiment, one or more further algorithmic step(s) are performed on the plurality of optimized nucleotide sequences in order to filter or remove optimized nucleotide sequences. The one or more further algorithmic step(s) may be referred to as motif screen, GC content analysis, and codon adaptation index (CAI) analysis.

Typically, a plurality of codon-optimized nucleotide sequences for use in the screening method of the invention ranges from 10-100 codon-optimized nucleotide sequences encoding the same protein of interest. For example, 10, 20, 30, 40, or 50 codon-optimized nucleotide sequences encoding the same protein of interest may be screened at the same time. In some embodiments, a screening method of the invention is performed with a plurality of nucleotide sequences encoding different proteins of interest. For example, the plurality of nucleotide sequences may include a first set of nucleotide sequences encoding a first protein of interest and a second set of nucleotide sequences encoding a second protein of interest. In some embodiments, there may be further sets of nucleotide sequences (e.g., a third, fourth and fifth set) encoding further proteins of interest (e.g., a third, fourth, and fifth protein of interest).

In some instances, a plurality of nucleotide sequences may include 50, 70, 90, 100, 200 or 300 nucleotide sequences, representing multiple sets of nucleotide sequences encoding different proteins of interest. Each set may comprise 10 to 30 different codon-optimized nucleotide sequences encoding the same protein of interest. In a specific embodiment, over 90 (e.g., 96) or over 300 (e.g., 384) nucleotide sequences may be screened at the same time using a screening method of the invention.

Filtering the plurality of codon-optimized sequences using the methods described herein produces an updated list of nucleotide sequences containing more effective sequences than if that same certain number of sequences were randomly selected from the list. The efficiency and reduction in complexity achieved by filtering does not, therefore, come at the cost of sacrificing a large number of effective optimized nucleotide sequences. For example, in certain embodiments, nucleotide sequences comprising termination signals the presence of which may lead to premature termination of in vitro transcription are removed. The absence of termination signals facilitates the synthesis of full-length mRNA transcripts from the nucleotide sequences using in vitro transcription.

Expressed more broadly, filtering the plurality of nucleotide sequences generated by a codon optimization algorithm identifies and removes nucleotide sequences failing to meet one or more criteria. The criteria may each relate to a certain further algorithmic step as described herein. In other words, the criteria may comprise retaining only nucleotide sequences that (a) lack a termination signal (a first criterion), (b) have a guanine-cytosine content within a predetermined guanine-cytosine content range (a second criterion), and (c) have a codon adaptation index greater than a predetermined codon adaptation index threshold (a third criterion). It will be appreciated that the numbering of the criteria used is for the sake of clarity only and is not intended to be limiting on the order of the steps, which is described in greater detail elsewhere herein. It will be appreciated that although specific criteria are described in detail herein, these may not be the only criteria for which the plurality of nucleotide sequences is screened.

### Motif Screen

In some embodiments, a motif screen filter may be applied to the plurality of nucleotide sequences generated by a codon optimization algorithm. In such embodiments, the plurality of nucleotide sequences is analyzed to determine whether each nucleotide sequence contains a termination signal. Any codon-optimized nucleotide sequence that contains one or more termination signal may be removed.

In some embodiments, a termination signal may have the nucleotide sequence: 5′-X₁ATCTX₂TX₃-3′, wherein X₁, X₂ and X₃ are independently selected from A, C, T or G. In a specific embodiment, a termination signal may have one of the following nucleotide sequences: TATCTGTT; TTTTTT; AAGCTT; GAAGAGC; and TCTAGA. The motif screen filter may determine whether each codon-optimized nucleotide sequence contains one, some, or all of these termination signals.

Each optimized nucleotide sequence may be analyzed in its entirety, i.e., from the first nucleotide in the sequence to the last nucleotide in the sequence. In a particular embodiment, the analysis of a certain optimized nucleotide sequence may stop when the presence of a termination signal is determined in that sequence; that sequence may then be removed without analyzing every one of its nucleotides. In the particular embodiment, this form of analysis may be applied to each codon-optimized nucleotide sequence in the plurality of nucleotide sequences. Analysis in this way can be advantageous because it is computationally efficient not to analyze an entire sequence if the presence of a termination signal in that sequence has already been determined. The additional motif-screen(s) yield(s) a subset of optimized nucleotide sequences that can be subjected to further analysis steps.

### GC Content Analysis

In some embodiments, a guanine-cytosine (GC) content filter may be applied to the plurality of codon-optimized nucleotide sequences. In such embodiments, the plurality of nucleotide sequences is analyzed to determine a GC content of each of the nucleotide sequences. As used herein, the GC content of a sequence is the percentage of bases in the nucleotide sequence that are guanine (G) or cytosine (C). Any optimized nucleotide sequence that has a GC content falling outside a predetermined GC content range (e.g., 40%-60%) may be removed from the plurality of nucleotide sequences generated by the codon optimization algorithm.

Each optimized nucleotide sequence may be analyzed in its entirety, i.e., from the first nucleotide in the sequence to the last nucleotide in the sequence. The GC content of the entire optimized nucleotide sequence may then be determined and sequences removed accordingly.

In some embodiments, only a portion of each optimized nucleotide sequence is analyzed and the GC content of that portion determined. In such embodiments, if the GC content of the analyzed portion falls outside the predetermined GC content range, the nucleotide sequence having that portion is removed from the list.

In a particular embodiment, the GC content filter is applied to each nucleotide sequence portion by portion, with the filter halting and the sequence being removed if a portion is determined to have a GC content falling outside the predetermined range. Analysis in this way can be advantageous because it is computationally efficient not to analyze an entire sequence if the presence of a portion in that sequence having a GC content falling outside the predetermined GC content range has already been found.

In a particular embodiment, the portions are non-overlapping, however, in other embodiments, the portions may overlap. It will be appreciated that this particular embodiment can be performed with any length of portion, for example, 5 to 300 nucleotides, or 10 to 200 nucleotides, or 15 to 100 nucleotides, or 20 to 50 nucleotides, or, in particular, 30 nucleotides or 100 nucleotides. In some embodiments, the predetermined GC content range may be selectable by a user. It will also be appreciated that this particular embodiment can be performed with any length of optimized nucleotide sequence.

### Codon Adaptation Index (CAI) Analysis

In some embodiments, a codon adaptation index (CAI) analysis may be performed on the plurality of nucleotide sequences. In such embodiments, the nucleotide sequences are analyzed to determine their CAI, wherein CAI is a measure of codon usage bias and can take a value of 0 to 1. Any nucleotide sequence having a CAI less than or equal to a predetermined CAI threshold may be removed from the plurality of nucleotide sequences generated by the codon optimization algorithm.

In some embodiments, the CAI threshold is 0.7, 0.75, 0.85, or 0.9. In a particular embodiment, the CAI threshold is 0.8.

A CAI may be calculated, for each optimized nucleotide sequence, in any way that would be apparent to a person skilled in the art, for example, as described in “The codon adaptation index—a measure of directional synonymous codon usage bias, and its potential applications” (Sharp and Li, 1987. Nucleic Acids Research 15(3), p. 1281-1295; available online at www.ncbi.nlm.nih.gov/pmc/articles/PMC340524/).

### Providing DNA Fragments of a Nucleotide Sequence of Interest

Processing of Nucleotide Sequences into DNA Fragments

If required, each of the nucleotide sequences provided in step (a) of a screening method of the invention may be divided into two or more DNA fragments with homologous ends. The homologous ends can be required for the assembly of the plasmid formed in step (d) of the screening method. Fragmentation can be performed manually, or by a fragmentation algorithm. In each instance, overlapping DNA fragments are generated, whereby the overlapping regions are the homologous ends required for assembly of the fragments. The overlapping regions (homologous ends) can be 15 to 40 base pairs long. More typically, the overlapping regions will be 15 to 30 base pairs long, e.g., 15 to 20 base pairs long.

The two or more DNA fragments are typically about 1000 base pairs long. In some embodiments, the two or more DNA fragments are at least 1000 base pairs long. For example, each of the two or more DNA fragments may be 1000 base pairs to 4000 base pairs long.

Error-free DNA fragments sized from 1000 base pairs to 4000 base pairs can be readily provided by chemical synthesis. The use of short DNA fragments may be advantageous, e.g., because they can typically be delivered within a few business days from submitting an order to an external provider. Moreover, they may be more readily assembled into larger constructs using commercially available molecular cloning kits. The inventors have found that a DNA fragment size of about 1000 base pairs can be particularly advantageous in the screening methods of the invention.

### Chemical Synthesis

Each of the two or more DNA fragments or the insert, as applicable, may be provided by a chemical synthesis process. In some embodiments, the two or more DNA fragments or the insert is about 1000 base pairs long. In other embodiments, the two or more DNA fragments or the insert is at least 1000 base pairs long. For example, each of the two or more DNA fragments or the insert may be about 900 base pairs to about 4000 base pairs long. In a particular embodiment, each of the two or more DNA fragments or the insert may be about 1000 base pairs to about 3000 base pairs long.

In certain embodiments, the chemical synthesis process that is used to provide the two or more DNA fragments has a median error rate of less than or equal to 1 error per 5000 base pairs. In particular embodiments, the chemical synthesis process has a median error rate of less than or equal to 1 error per 10,000 base pairs.

Many commercial suppliers provide chemically synthesized DNA fragments that meet the above requirements. For example, Integrated DNA Technologies (IDT) provides “gBlock” DNA fragments of 1000 base pairs in length with a median error rate of 1 error per 5000 base pairs at an amount of 1000 ng, which are ready for shipping within 3-5 days. IDT's “gBlock HiFi” DNA fragments are provided at a length of 1000-3000 base pairs at a median error rate of 1 error per 12,000 base pairs. Other commercially available products include GeneArt's “Strings” and “High-Q Strings” DNA fragments with lengths of up to 3000 base pairs and 1200 base pairs, respectively, at median error rates of less 1 error per 5000 base pairs and less than 1 error per 10,000 base pairs, respectively.

Due to advances in the ability to accurately, reliably and rapidly synthesize high-quality long DNA fragments at scale, the insert comprising the nucleotide sequence of interest may be provided without the need to generate two or more DNA fragments. For example, it has become possible to synthesize DNA at an error rate of 1:68,000 that is up to 7,000 base pairs in length. Indeed, using sophisticated microfluidic technology, DNA of 20,000 base pairs in length or more can be synthesized at very low error rates. Therefore, in some embodiments, the insert may be about 1,000 base pairs to about 20,000 base pairs long. In particular embodiments, the insert may be about 1,000 base pairs to about 8,000 base pairs long. In specific embodiments, the insert may be about 1,000 base pairs to about 7,000 base pairs long. In some embodiments, the chemical synthesis process used to prepare the inserts has a median error rate of less than or equal to 1 error per 20,000 base pairs, e.g., less than or equal to 1 error per 30,000 base pairs. In particular embodiments, the chemical synthesis process used to prepare the inserts has a median error rate of less than or equal to 1 error per 40,000 base pairs, e.g., less than or equal to 1 error per 50,000 base pairs.

### Providing the Vector Backbone

Step (c) of a screening method of the invention provides a vector backbone for receiving an insert comprising a codon-optimized nucleotide sequence of interest. Assembly of the vector backbone and the insert yields a plasmid that serves as a template for the in vitro transcription of the nucleotide sequence into an mRNA transcript in step (e) of the screening method.

The vector backbone typically comprises a positive selection marker gene. A positive selection marker gene can encode a gene product (e.g., a protein or enzyme) that provides resistance to an antibiotic, enabling the selection of bacterial colonies (e.g., Escherichia coli) carrying a plasmid comprising the nucleotide sequence on growth medium containing the antibiotic. Commonly used positive selection markers include kanamycin resistance and ampicillin resistance genes.

In some embodiments, the vector backbone can also comprise a negative selection marker gene. Typically, negative selection marker genes encode gene product that impedes growth or survival of a bacterial organism (e.g., Escherichia coli). A negative selection marker is commonly placed in the vector backbone in such a manner that the presence of an insert comprising a nucleotide sequence of interest either displaces the marker gene or disrupts its function such that bacterial colonies carrying a plasmid comprising the nucleotide sequence of interest survive, while those without the plasmid perish. A commonly used negative selection marker gene is the ccdB gene, which encodes the CcdB toxin.

The vector backbone commonly also comprises an origin of replication. The origin of replication enables the resulting plasmid to be maintained in a bacterial organism (e.g., Escherichia coli). It also controls the copy number within the bacterial cell. The inventors have found that a vector backbone comprising an origin of replication having the nucleotide sequence of SEQ ID NO: 2 is particularly suitable for use with the screening methods of the invention. Without wishing to be bound by any particular theory, the inventors believe that this origin of replication comprising a stabilizing mutation for maintenance of plasmids with nucleotide sequence of interest (typically codon-optimized mammalian gene sequences, in particular human gene sequences) in bacterial cells (specifically, Escherichia coli).

The vector backbone may contain additional elements such as a 5′ untranslated region (5′ UTR), a 3′ untranslated region (3′ UTR), an RNA polymerase promoter and, optionally, a poly(A) sequence or a poly(C) sequence. If present in the vector backbone, these elements are arranged in such a manner that insertion of the nucleotide sequence links them operationally. In particular, the 5′ UTR, 3′UTR and the optional poly(A) or poly(C) sequence are arranged in such a manner that the 5′ UTR is positioned at the 5′ end of the nucleotide sequence and the 3′ UTR and the optional poly(A) or poly(C) sequence are positioned at the 3′ end of the nucleotide sequence, respectively. Moreover, the RNA polymerase promoter is positioned upstream of the 5′ UTR such that adding the resulting plasmid comprising the nucleotide sequence in an in vitro transcription reaction mixture under appropriate reaction conditions results in the synthesis of an mRNA transcript comprising the 5′ UTR, the nucleotide sequence, the 3′ UTR and, optionally, the poly(A) or poly(C) sequence.

In some embodiments, the vector backbone is provided by linearizing a plasmid, for example, by using a suitable restriction enzyme. The plasmid is linearized in such manner that the vector backbone is ready to receive an insert comprising a nucleotide sequence of interest so as to form a suitable template for in vitro transcription of an mRNA transcript comprising the nucleotide sequence. For example, if the vector backbone comprises the elements described in the preceding paragraph, it is provided in such a manner that insertion of the nucleotide sequence yields a plasmid comprising the nucleotide sequence flanked by the 5′ UTR and the 3′ UTR and operationally linked to an RNA polymerase promoter.

In some embodiments, the vector backbone is provided using polymerase chain reaction (PCR). In these embodiments, a plurality of primer pairs encoding the second and third sets of homologous ends or the first and second sets of homologous ends, as applicable, are provided. The provided homologous ends allow the assembly of the vector backbone with the DNA fragments or inserts comprising corresponding homologous ends. In the presence of the primer pairs, a DNA polymerase, and a template plasmid comprising the vector backbone, the PCR can be performed to provide copies of the vector backbone for use in step (c) of a screening method of the invention.

The use of PCR may be advantageous for two reasons. The PCR reaction mixture may be used directly in the assembly step (d) without any purification step. In addition, the use of PCR makes it possible to provide the vector backbone as vector fragments without having to redesign the source plasmid to provide suitable restriction sites. For example, primer pairs may be designed in such a manner that PCR amplification yields two or more vector fragments with overlapping ends (i.e., homologous ends) that, when assembled via these ends, results in the vector backbone.

### Homologous Ends

Joining two or more DNA fragments together to form the nucleotide sequence encoding a protein of interest that was generated by a codon optimization algorithm relies on homologous ends that are present in each of the DNA fragments. Typically, the DNA fragments are designed to comprise homologous ends that are about 15 base pairs to about 30 base pairs long.

For example, a nucleotide sequence may be divided up into three DNA fragments A, B and C that when assembled in this order form the nucleotide sequence. DNA fragment A on its 3′ end comprises a nucleic acid sequence 1 that is homologous to nucleic acid sequence 1′ found at the 5′ end of DNA fragment B. DNA fragment B on its 3′ end comprises a nucleic acid sequence 2 that is homologous to nucleic acid sequence 2′ found at the 5′ end of DNA fragment C. Nucleic acid sequences 1 and 2 and nucleic acid sequences 1′ and 2′ are a first set of homologous ends. In addition, DNA fragment A comprises a nucleic acid sequence 3 at its 5′ end that is homologous to nucleic acid sequence 3′ found at the 3′ end of the vector backbone. DNA fragment C comprises a nucleic acid sequence 4 at its 3′ end that is homologous to nucleic acid sequence 4′ found at the 5′ end of the vector backbone. Nucleic acid sequences 3 and 4 and nucleic acid sequences 3′ and 4′ are a second set of homologous ends. Assembly via the first set of homologous ends yields an insert comprising the nucleotide sequence. The insert comprises homologous ends from the second set such that assembly of the insert and the vector backbone via the second set of homologous ends yields a plasmid. In some embodiments, the second set of homologous ends comprises a first polylinker sequence (comprised within nucleic acid sequences 3 and 3′, respectively) and a second polylinker sequence (comprised within nucleic acid sequences 4 and 4′, respectively).

In some embodiments, the vector backbone itself is provided as multiple vector fragments. For example, the vector backbone may be divided up into three vector fragments D, E and F that when assembled in this order form the vector backbone. Vector fragment D on its 3′ end comprises a nucleic acid sequence I that is homologous to nucleic acid sequence I′ found at the 5′ end of vector fragment E. Vector fragment E on its 3′ end comprises a nucleic acid sequence II that is homologous to nucleic acid sequence II′ found at the 5′ end of vector fragment F. Nucleic acid sequences I and II and nucleic acid sequences I′ and II′ are a third set of homologous ends. In addition, vector fragment D comprises a nucleic acid sequence 4′ at its 5′ end that is homologous to nucleic acid sequence 4 found at the 3′ end of the insert. Vector fragment F comprises a nucleic acid sequence 3′ at its 3′ end that is homologous to nucleic acid sequence 3 found at the 5′ end of the insert. Accordingly, when assembled via the third set of homologous ends, vector fragments D, E and F yield a vector backbone with the second set of homologous ends. As described in the preceding paragraph, assembly of the insert and the vector backbone via the second set of homologous ends yields a plasmid. In some embodiments, the second set of homologous ends comprises a first polylinker sequence (comprised within nucleic acid sequences 3 and 3′, respectively) and a second polylinker sequence (comprised within nucleic acid sequences 4 and 4′, respectively).

### Assembly Process

Assembly of DNA fragments (insert) and vector fragments (vector backbone) via homologous ends (as described in the preceding section) in step (d) of a screening method of the invention can be done in a variety of ways. In a typical embodiment, step (d) is performed in the presence of a DNA polymerase and/or an exonuclease, and optionally a ligase. In some embodiments, the 5′ exonuclease is part of the DNA polymerase, i.e., a suitable DNA polymerase with exonuclease function is provided. In other embodiments, a 5′ exonuclease and a DNA polymerase are provided separately. In a particular embodiment, step (d) is performed in the presence of a 5′ exonuclease, a DNA polymerase and a ligase. In some embodiments, the presence of ligase is optional, i.e., only the presence of a 5′ exonuclease and a DNA polymerase may be required. Without wishing to be bound by any particular theory, it is thought that the assembled nucleic acid fragments can be ligated by an endogenous ligase following transformation of the assembled plasmid into a bacterial cell (Benoit et al. Seamless insert-plasmid assembly at high efficiency and low cost, PLoS One 2016; 11(4), p. e0153158).

Assembly methods utilizing these enzyme reagents for the assembly of DNA fragments via homologous ends are known (see, for example, Casini et al. Bricks and blueprints: methods and standards for DNA assembly, Nat. Rev. Mol. Cell Biol. 2015, 16, p. 568-576). Sequence homology-based methods join DNA fragments that share homologous ends. Homologous ends can be 15 base pairs to 40 base pairs long. More typically, the homologous ends are 15 to 30 base pairs long. One benefit of homology-based methods is that there is no requirement to remove any restriction sites from within the sequences to be joined.

A well-known exemplary homology-based DNA assembly method is Gibson assembly (Gibson et al. Enzymatic assembly of DNA molecules up to several hundred kilobases. Nat. Methods 2009, 6, p. 343-345). Gibson assembly employs a “chew-back” mechanism on one strand of the double-stranded DNA to create complementary single-stranded overhangs. It utilizes a 5′ exonuclease, a DNA polymerase and a ligase for assembly of DNA fragments. Other homology-based DNA assembly methods may be employed to perform step (d) of a screening method of the invention. Other suitable methods include, for example, Circular Polymerase Extension Cloning (CPEC), Overlap Extension PCR (OE-PCR), Uracil-Specific Excision Reagent (USER) assembly and enzymatic digestion which can be used to convert the homologous ends of the DNA fragments/insert/vector fragments/vector backbone into single-strand overhangs that can directly anneal.

Particularly useful methods for performing step (d) of a screening method of the invention include Sequence and Ligation Independent Cloning (SLIC), Gibson assembly, CPEC (circular polymerase extension cloning) and SLiCE (Seamless Ligation Cloning Extract). Various commercial cloning kits are available which assemble nucleic acid fragments via homologous ends without requirements for restriction enzymes, and no scar sequences between fragments. These include GeneArt® Seamless Cloning and Assembly (Thermo Fisher Scientific), NEBuilder® HiFi DNA Assembly (NEB), Cold Fusion Cloning (System Biosciences), and In-fusion Cloning (Clontech).

In a specific embodiment, a 5′ exonuclease recesses the double-stranded DNA fragment from the 5′ ends to generate single-stranded complementary overhangs out of the homologous ends. These complementary overhangs of the homologous ends anneal, and a DNA polymerase and optionally a ligase (e.g., a Taq DNA ligase) can (optionally covalently) join the complementary overhangs together to assemble the DNA fragments into an insert. In the absence of a ligase, the DNA fragments are thought to be ligated (and thus covalently joined) by an endogenous ligase following transformation of the assembled plasmid into bacterial cells. In some embodiments, the 5′ exonuclease is part of the DNA polymerase (e.g., a T4 DNA polymerase). In other embodiments, the 5′ exonuclease (e.g., a T5 exonuclease) is provided as a separate enzyme, e.g., in addition to the DNA polymerase (e.g., a Phusion Flash® DNA polymerase). In a typical embodiment, the 5′ exonuclease (e.g., a T5 exonuclease) and DNA polymerase (e.g., a fusion DNA polymerase) do not compete, which allows simultaneous activity in a single isothermal reaction. In some embodiments, step (d) of a screening method of the invention is performed at a temperature of about 40° C. to about 60° C. In a specific embodiment, step (d) is an isothermal reaction performed at about 50° C. In particular embodiments, step (d) of a screening method of the invention is performed over a period of about 15 minutes to about 60 minutes.

In a typical embodiment, the 5′ exonuclease generates complementary single-stranded overhangs out of the homologous ends of the DNA fragments. The complementary single-stranded overhangs anneal and are joined (optionally covalently through the action of a ligase) to form the insert with single-stranded overhangs complementary to single-stranded overhangs of a vector backbone. The 5′ exonuclease also generates single-stranded overhangs out of the homologous ends of the vector backbone, which are complementary to the single-stranded overhangs of the insert. The complementary single-stranded overhangs of the insert and vector backbone anneal and are joined (optionally covalently through the action of a ligase) to introduce the insert (formed from the two or more DNA fragments) into the vector backbone.

In some embodiments, the vector backbone itself is assembled from vector fragments with homologous ends. In these embodiments, the 5′ exonuclease generates complementary single-stranded overhangs out of the homologous ends of the vector fragments. The complementary single-stranded overhangs anneal and are joined (optionally covalently by the action of a ligase) to form a vector backbone with single-stranded overhangs complementary to the single-stranded overhangs of the insert.

In a specific embodiment, an assembly method for use with the invention employs a T5 exonuclease, a fusion DNA polymerase and a Taq DNA ligase.

### Plasmid Component

The assembly of the insert (optionally formed from two or more DNA fragments) and the vector backbone (optionally formed from two or more vector fragments) yields a plasmid. The nucleotide sequence in the assembled plasmid is flanked by a 5′ untranslated region (5′ UTR) and a 3′ untranslated region (3′ UTR) and operationally linked to an RNA polymerase promoter. The 5′ UTR, the 3′ UTR and the RNA polymerase promoter are either part of the insert or the vector backbone

### 5′ Untranslated Region

A plasmid for use with a screening method of the invention comprises a nucleotide sequence encoding a 5′ UTR operably linked to the optimized nucleotide sequence. In particular embodiments, the 5′ UTR is different from the 5′ UTR of a naturally occurring mRNA encoding the amino acid sequence. In a specific embodiment, the 5′ UTR has the nucleotide sequence of SEQ ID NO: 9.

### 3′ Untranslated Region

A plasmid for use with a screening method of the invention comprises a nucleotide sequence encoding a 3′ UTR operably linked to a nucleotide sequence of interest. In particular embodiments, the 3′ UTR is different from the 3′ UTR of a naturally occurring mRNA encoding the amino acid sequence. In a specific embodiment, the 3′ UTR has the nucleotide sequence of SEQ ID NO: 10 or SEQ ID NO: 11.

### RNA Polymerase Promoter

A plasmid for use with a screening method of the invention comprises an RNA polymerase promoter. A nucleotide sequence of interest inserted in the plasmid is operationally linked to the RNA polymerase promoter. In the presence of an RNA polymerase, e.g., an SP6 RNA polymerase or a T7 RNA polymerase, in an in vitro transcription mixture, the nucleotide sequence is transcribed into an mRNA transcript.

Any promoter that can be recognized by an SP6 RNA polymerase may be used in a screening method of the invention. Typically, an SP6 promoter comprises 5′-ATTTAGGTGACACTATAG-3′ (SEQ ID NO: 12). Variants of the SP6 promoter have been discovered and/or created to optimize recognition and/or binding of SP6 to its promoter. Non-limiting variants include, but are not limited to:

| (SEQ ID NO: 13) | |
|---|---|
| | 5′-ATTTAGGGGACACTATAGAAGAG-3′; |
| (SEQ ID NO: 14) | |
| | 5′-ATTTAGGGGACACTATAGAAGG-3′; |
| (SEQ ID NO: 15) | |
| | 5′-ATTTAGGGGACACTATAGAAGGG-3′; |
| (SEQ ID NO: 16) | |
| | 5′-ATTTAGGTGACACTATAGAA-3′; |
| (SEQ ID NO: 17) | |
| | 5′-ATTTAGGTGACACTATAGAAGA-3′; |
| (SEQ ID NO: 18) | |
| | 5′-ATTTAGGTGACACTATAGAAGAG-3′; |
| (SEQ ID NO: 19) | |
| | 5′-ATTTAGGTGACACTATAGAAGG-3′; |
| (SEQ ID NO: 20) | |
| | 5′-ATTTAGGTGACACTATAGAAGGG-3′; |
| (SEQ ID NO: 21 | |
| | 5′-ATTTAGGTGACACTATAGAAGNG-3′; |
| | and |
| (SEQ ID NO: 22) | |
| | 5′-CATACGATTTAGGTGACACTATAG-3′. |

Where N is used in the nucleotide sequences, N is A, C, T or G.

In addition, a suitable SP6 promoter may be about 95%, 90%, 85%, 80%, 75%, or 70% identical or homologous to any one of SEQ ID NO: 13 to SEQ ID NO: 22. Moreover, a suitable SP6 promoter may include one or more additional nucleotides 5′ and/or 3′ to any of the promoter sequences described herein.

Alternatively, any promoter that can be recognized by a T7 RNA polymerase may be used in the screening methods of the invention. Typically, a T7 promoter comprises 5′-TAATACGACTCACTATAG-3′ (SEQ ID NO: 23).

### Poly(A)/Poly(C) Sequence

In certain embodiments, a plasmid for use with a screening method of the invention comprises a poly(A) sequence or a poly(C) sequence at the 3′ end of the 3′UTR. Accordingly, in vitro transcription of the nucleotide sequence inserted in the plasmid yields an mRNA transcript with a poly(A) or poly(C) tail on the 3′ terminus.

A poly(A) or poly(C) tail encoded by a poly(A) sequence or a poly(C) sequence in the plasmid typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively.

In some embodiments, a poly(A) or poly(C) tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively.

In some embodiments, a poly(A) sequence or a poly(C) sequence includes a combination of poly(A)s and poly(C)s with various lengths as described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### Termination Signal

The presence of the two more termination signals at the 3′ end may avoid the need for linearization of the plasmid prior to in vitro transcription of the nucleotide sequence in step (e) of a screening method of the invention.

Accordingly, in certain embodiments, a plasmid for use with a screening method of the invention comprises two or more termination signals arranged sequentially and positioned at the 3′ end of the 3′UTR. In a specific embodiment, the plasmid comprises three termination signals. In embodiments in which the plasmid comprises a poly(A) sequence or a poly(C) sequence, the two or more termination signals are positioned at the 3′ end of the poly(A) sequence or poly(C) sequence.

In a typical embodiment, each termination signal is separated by 10 base pairs or fewer. For example, each termination signal may be separated by 5-10 base pairs.

In some embodiments, the two or more termination signals comprise the following nucleotide sequence: 5′-X₁ATCTX₂TX₃-3′, wherein X₁, X₂ and X₃ are independently selected from A, C, T or G. In particular embodiments, the two or more termination signals comprise the nucleic acid sequence 5′-X₁ATCTGTT-3′. In one specific embodiment, X₁ is T. In another specific embodiment, X₁ is C. In further specific embodiments, the two or more termination signals comprise one of the following nucleotide sequences: TATCTGTT; and/or TTTTTT; and/or AAGCTT; and/or GAAGAGC; and/or TCTAGA. In particular embodiments, the two or more termination signals are selected from 5′-TTTTATCTGTTTTTTT-3′ (SEQ ID NO: 3), 5′-TTTTATCTTTTTTTTT-3′ (SEQ ID NO: 4), 5′-CGTTTTATCTGTTTTTTT-3′ (SEQ ID NO: 5), 5′-CGTTCCATCTGTTTTTTT-3′ (SEQ ID NO: 6, 5′-CGTTTTATCTGTTTGTTT-3′ (SEQ ID NO: 7), or 5′-CGTTTTATCTGTTGTTTT-3′ (SEQ ID NO: 8).

In some embodiments, the two or more termination signals are encoded by the following nucleotide sequence: (a) 5′-X₁ATCTX₂TX₃-(Z_{N})-X₄ATCTX₅TX₆-3′ or (b) 5′-X₁ATCTX₂TX₃-(Z_{N})-X₄ATCTX₅TX₆-(Z_{M})-X₇ATCTX₈TX₉-3′, wherein X₁, X₂, X₃, X₄, X₅, X₆, X₇, X₈ and X₉ are independently selected from A, C, T or G, Z_{N} represents a spacer sequence of N nucleotides, and Z_{M} represents a spacer sequence of M nucleotides, each of which are independently selected from A, C, T or G, and wherein N and/or M are independently 10 or fewer. For example, N can be 5, 6, 7, 8, 9 or 10. M can be 5, 6, 7, 8, 9 or 10. Z can be T. In one specific embodiment, a plasmid for use with a screening method of the invention comprises the following sequence at its 3′ end of the 3′UTR: TTTTATCTGTTTTTTTTTTTTTATCTGTTTTTTTTT (SEQ ID NO: 24). The core motif of the termination signal is underlined. In another specific embodiments, a plasmid for use with a screening method of the invention comprises the following sequence at its 3′ end of the 3′UTR: TTTTATCTGTTTTTTTTTTTTTATCTGTTTTTTTTTTTTTATCTGTTTTTTTTT (SEQ ID NO: 25). The core motif of the termination signal is underlined.

### Bacterial Terminator

In some embodiments, a plasmid for use with a screening method of the invention comprises a bacterial terminator. The bacterial terminator is located in the vector backbone upstream of the RNA polymerase promoter in such a manner that it prevents transcription of the optimized nucleotide sequences by a bacterial RNA polymerase.

Accordingly, a plasmid is provided herein which comprises a vector backbone and an insert comprising an optimized nucleotide sequence encoding a therapeutic protein, wherein (a) the optimized nucleotide sequence is flanked by a 5′ untranslated region (5′ UTR) and a 3′ untranslated region (3′ UTR) and operationally linked to an RNA polymerase promoter; and (b) a bacterial terminator is located in the vector backbone upstream of the RNA polymerase promoter, wherein the bacterial terminator prevents transcription of the optimized nucleotide sequences by a bacterial RNA polymerase.

In some embodiments, the RNA polymerase promoter is an SP6 RNA polymerase promoter. In a specific embodiment, the bacterial RNA polymerase is an endogenous Escherichia coli RNA polymerase.

In some embodiments, the bacterial terminator is an Escherichia coli ropC terminator. A suitable Escherichia coli ropC is found in the genome of Escherichia coli strain JME66 (GenBank accession no.: CP042844.1; the region comprising nucleotides 1660501 to 1660620). In a specific embodiment, the bacterial terminator has the following sequence:

(SEQ ID NO: 28)
GGCGCCCTTAAATATTCTGACAAATGCTCTTTCCCTAAACTCCCCCCATA
AAAAAACCCGCCGAAGCGGGTTTTTACGTTATTTGCGGATTAACGATTAC
TCGTTATCAGAACCGCCCAG.

In some embodiments, the bacterial terminator is a Staphylococcus aureus bla terminator. A suitable Staphylococcus aureus bla terminator is found in the genome of Staphylococcus aureus strain 16405 (GenBank accession no.: CP053354.1; the region comprising nucleotides 26736 to 27036). In a specific embodiment, the bacterial terminator has the following sequence:

(SEQ ID NO: 29)
TATTCTAAATGCATAATAAATACTGATAACATCTTATAGTTTGTATTATA
TTTTGTATTATCGTTGACATGTATAATTTTGATATCAAAAACTGATTTTC
CCTTTATTATTTTCGAGATTTATTTTCTTAATTCTCTTTAACAAACTAGA
AATATTGTATATACAAAAAATCATAAATAATAGATGAATAGTTTAATTAT
AGGTGTTCATCAATCGAAAAAGCAACGTATCTTATTTAAAGTGCGTTGCT
TTTTTCTCATTTATAAGGTTAAATAATTCTCATATATCAAGCAAAGTGAC
A

### Polylinkers

In some embodiments, it is advantageous to provide one or more polylinker sequences (multiple cloning sites) in a plasmid for use with a screening method of the invention. For example, a plasmid may comprise a first polylinker sequence and a second polylinker sequence that frame an insert comprising a nucleotide sequence of interest and thus make it possible to confirm by restriction digest the presence of the insert in the plasmid based on its size. The first and second polylinkers may be included in the set of homologous ends that are used for assembly of the insert and the vector backbone into a plasmid.

In some embodiments, the homologous ends are chosen such that in the assembled plasmid the first polylinker sequence is located immediately upstream of the RNA polymerase promoter, and the second polylinker sequence is located immediately downstream of the 3′ UTR.

In some embodiments, a plasmid for use with a screening method of the invention comprises a bacterial terminator. The bacterial terminator is located in the vector backbone of the plasmid upstream of the RNA polymerase promoter in such a manner as to prevent transcription of the nucleotide sequence by a bacterial RNA polymerase. In such embodiments, a first polylinker sequence may be located between the bacterial terminator and the RNA polymerase promoter, and a second polylinker sequence may located immediately downstream of the 3′ UTR.

### Exemplary Plasmids

In exemplary embodiments, a screening method of the invention may make use of a plasmid comprising (i) a vector backbone and (ii) an insert comprising an optimized nucleotide sequence encoding a therapeutic protein, wherein (a) the optimized nucleotide sequence is flanked by a 5′ untranslated region (5′ UTR) and a 3′ untranslated region (3′ UTR) and operationally linked to an RNA polymerase promoter; and (b) a bacterial terminator is located in the vector backbone upstream of the RNA polymerase promoter. The presence of the bacterial terminator prevents transcription of the optimized nucleotide sequences by a bacterial RNA polymerase. This is advantageous for the stable maintenance and propagation of the plasmid in a bacterial cell.

For example, Escherichia coli is commonly used to amplify a plasmid after assembly and prior to the in vitro transcription reaction, or in order to obtain large amounts of plasmid DNA for manufacturing of a therapeutic mRNA comprising the optimized nucleotide sequence. Accordingly, in a typical embodiment, the bacterial RNA polymerase is an endogenous Escherichia coli RNA polymerase.

The optimized nucleotide sequence is typically generated by a codon optimization algorithm as described herein. In a typical embodiment, the optimized nucleotide sequence is codon optimized. In a specific embodiment, the optimized nucleotide sequence consists of codons associated with a usage frequency which is greater than or equal to 10%. Moreover, the optimized nucleotide sequence typically has one or more of the following additional features: (i) it does not contain a termination signal having the following nucleotide sequence: 5′-X₁ATCTX₂TX₃-3′, wherein X₁, X₂ and X₃ are independently selected from A, C, T or G; (ii) it does not contain any negative cis-regulatory elements and negative repeat elements; (iii) it has a codon adaptation index greater than 0.8; and (iv) each portion of the optimized nucleotide sequence has a guanine cytosine content range of 30%-70%, when divided into non-overlapping 30 nucleotide-long portions. In a particular embodiment, the optimized sequence is codon optimized and additionally comprises all of features (i)-(iv). In specific embodiments, the optimized nucleotide sequence does not contain a termination signal having one of the following sequences: TATCTGTT; TTTTTT; AAGCTT; GAAGAGC; and TCTAGA.

In some embodiments, the bacterial terminator is an Escherichia coli ropC terminator. In other embodiments, the bacterial terminator is a Staphylococcus aureus bla terminator.

In some embodiments, the RNA polymerase promoter is an SP6 RNA polymerase promoter. SP6 RNA polymerase has been shown to provide high yields of full-length mRNA transcripts during large-scale manufacturing of therapeutic mRNA.

In some embodiments, the plasmid further comprises a first polylinker sequence and a second polylinker sequence, wherein the first polylinker sequence is located between the bacterial terminator and the RNA polymerase promoter, and a second polylinker sequence is located between the optimized nucleotide sequence and the 3′ UTR.

In some embodiments, the vector backbone comprises an origin of replication having the nucleotide sequence of SEQ ID NO: 2.

The ultimate goal of a screening method of the invention can be to identify a plasmid which comprises an optimized nucleotide sequence encoding a therapeutic protein of interest and can be used for the large-scale production of a therapeutic mRNA. Accordingly, in some embodiments, the plasmid is used in a method of manufacturing a therapeutic mRNA. Such a method may comprise adding the plasmid to an in vitro transcription reaction mixture to transcribe the nucleotide sequence into mRNA transcripts; and purifying the mRNA transcripts. Suitable reaction mixtures and conditions for in vitro transcription of the mRNA transcripts from the plasmid are described elsewhere herein. Purification may be done as described elsewhere herein by precipitating the mRNA transcripts through the addition of (i) a chaotropic salt, and (ii) an alcohol or an amphiphilic polymer.

In some embodiments, the mRNA transcripts are subject to post-synthesis processing as described herein. Such post-synthesis processing may comprise one or more additional purification steps after each processing step. These additional purification steps may also be done as described elsewhere herein by precipitating the mRNA transcripts through the addition of (i) a chaotropic salt, and (ii) an alcohol or an amphiphilic polymer.

### In Vitro Transcription

In step (e) of a screening method of the invention, a plasmid is added to an in vitro transcription reaction mixture to transcribe the nucleotide sequence into an mRNA transcript. In vitro transcription (also referred to commonly as “in vitro synthesis”) can be typically performed with a linear or circular plasmid in a reaction mixture comprising a pool of ribonucleotide triphosphates, a buffer system that may include DTT and magnesium ions, and an appropriate RNA polymerase (e.g., T3, T7, or SP6 RNA polymerase),

In some embodiments, the plasmid is linearized before step (e) is performed. Linearization is typically performed by a restriction enzyme that recognizes a single cut site in the plasmid. Linearization results in a double-stranded DNA template in which the RNA polymerase promoter of the plasmid is operationally linked to the 5′ UTR, the nucleotide sequence of interest, the 3′ UTR, and optionally the poly(A) sequence or the poly(C) sequence so as to yield an mRNA transcript in the presence of an in vitro transcription mixture that includes these sequence elements in the specified order starting with the 5′ UTR.

In some embodiments, the mRNA transcript synthesized from the nucleic acid vector during in vitro transcription does not contain a poly(A) tail. A poly(A) tail may be added to the mRNA transcript in a post-synthesis processing step.

### Composition of the Reaction Mixture

In some embodiments, a concentration of 100 to 1000 Units/ml of the RNA polymerase (e.g., T7 RNA polymerase) may be used in an in vitro transcription reaction mixture as described herein. For example, a concentration of 300 to 500 Units/ml of a T7 RNA polymerase may be used in a screening method of the invention. In particular embodiments, the RNA polymerase (e.g., an SP6 RNA polymerase) in an in vitro transcription reaction mixture as described herein is at a concentration ranging from 0.01-0.1 mg/ml. For example, a concentration of about 0.1 mg/ml of SP6 RNA polymerase may be used in a screening method of the invention.

The concentration of each ribonucleotide (e.g., ATP, UTP, GTP, and CTP) in a reaction mixture is about 0.1 mM to about 10 mM, e.g., about 1 mM to about 10 mM, about 2 mM to about 10 mM, about 3 mM to about 10 mM, about 1 mM to about 8 mM, about 1 mM to about 6 mM, about 3 mM to about 10 mM, about 3 mM to about 8 mM, about 3 mM to about 6 mM, about 4 mM to about 5 mM. In some embodiments, each ribonucleotide is at about 5 mM in a reaction mixture. In some embodiments, the total concentration of rNTPs (for example, ATP, GTP, CTP and UTPs combined) used in the reaction range from 1 mM to 40 mM. In some embodiments, the total concentration of rNTPs (for example, ATP, GTP, CTP and UTPs combined) used in the reaction range from 1 mM to 30 mM, or 1 mM to 28 mM, or 1 mM to 25 mM, or 1 mM to 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

In a particular embodiment, the concentration of each rNTP in a reaction mixture is optimized based on the frequency of each nucleic acid in the nucleic acid sequence that encodes a given mRNA transcript. Specifically, such a sequence-optimized reaction mixture comprises a ratio of each of the four rNTPs (e.g., ATP, GTP, CTP and UTP) that corresponds to the ratio of these four nucleic acids (A, G, C and U) in the mRNA transcript.

In some embodiments, a start nucleotide is added to the reaction mixture before the start of the in vitro transcription. A start nucleotide is a nucleotide which corresponds to the first nucleotide of the mRNA transcript (+1 position). The start nucleotide may be especially added to increase the initiation rate of the RNA polymerase. The start nucleotide can be a nucleoside monophosphate, a nucleoside diphosphate, a nucleoside triphosphate. The start nucleotide can be a mononucleotide, a dinucleotide or a trinucleotide. In embodiments where the first nucleotide of the mRNA transcript is a G, the start nucleotide is typically GTP or GMP. In a specific embodiment, the start nucleotide is a cap analog. The cap analog may be selected from the group consisting of G[5′]ppp[5′]G, m⁷G[5′]ppp[5′]G, m₃^{2,2,7}G[5′]ppp[5′]G, m₂^{7,3′-O}G[5′]ppp[5′]G (3′-ARCA), m₂^{7,2′-O}GpppG (2′-ARCA), m₂^{7,2′-O}GppspG D1 (β-S-ARCA D1) and m₂^{7,2′-O}GppspG D2 (β-S-ARCA D2).

In specific embodiments, the first nucleotide of the RNA transcript is G, the start nucleotide is a cap analog of G and the corresponding rNTP is GTP. In such embodiments, the cap analog is present in the reaction mixture in an excess in comparison to GTP. In some embodiments, the cap analog is added with an initial concentration in the range of about 1 mM to about 20 mM, about 1 mM to about 17.5 mM, about 1 mM to about 15 mM, about 1 mM to about 12.5 mM, about 1 mM to about 10 mM, about 1 mM to about 7.5 mM, about 1 mM to about 5 mM or about 1 mM to about 2.5 mM.

More typically in the context of the present invention, a cap structure such as a cap analog is added to the mRNA transcripts obtained during in vitro transcription only after the mRNA transcripts have been synthesized, e.g., in a post-synthesis processing step. Typically, in such embodiments, the mRNA transcripts are first purified (e.g., by tangential flow filtration) before a cap structure is added.

The reaction mixture typically includes a salt/buffering agent, e.g., Tris, HEPES, ammonium sulfate, sodium bicarbonate, sodium citrate, sodium acetate, potassium phosphate, sodium phosphate, sodium chloride, and magnesium chloride. The pH of the reaction mixture may be from about 6 to 8.5, e.g., from 6.5 to 8.0 or, more typically, from 7.0 to 7.5. In some embodiments, the pH is 7.5.

### SP6 RNA Polymerase

In some embodiments, the mRNA is synthesized by a SP6 RNA polymerase. In some embodiments, the SP6 RNA polymerase is a naturally occurring SP6 RNA polymerase. In some embodiments, the SP6 RNA polymerase is a recombinant SP6 RNA polymerase. In some embodiments, the SP6 RNA polymerase comprises a tag. Tags can be used to facilitate protein detection or purification. In some embodiments, the tag is a his-tag, which, for example, can be used for purification with Ni-NTA affinity chromatography.

SP6 RNA polymerase is a DNA-dependent RNA polymerase with high sequence specificity for SP6 promoter sequences. Typically, SP6 RNA polymerase catalyzes the 5′-3′ in vitro synthesis of RNA on either single-stranded DNA or double-stranded DNA downstream from its promoter. SP6 RNA polymerase incorporates native ribonucleotides and/or modified ribonucleotides into the polymerized transcript.

The sequence for bacteriophage SP6 RNA polymerase was initially described (GenBank: Y00105.1) as having the following amino acid sequence:

(SEQ ID NO: 26)
MQDLHAIQLQLEEEMFNGGIRRFEADQQRQIAAGSESDTAWNRRLLSELI
APMAEGIQAYKEEYEGKKGRAPRALAFLQCVENEVAAYITMKVVMDMLNT
DATLQAIAMSVAERIEDQVRFSKLEGHAAKYFEKVKKSLKASRTKSYRHA
HNVAVVAEKSVAEKDADFDRWEAWPKETQLQIGTTLLEILEGSVFYNGEP
VFMRAMRTYGGKTIYYLQTSESVGQWISAFKEHVAQLSPAYAPCVIPPRP
WRTPFNGGFHTEKVASRIRLVKGNREHVRKLTQKQMPKVYKAINALQNTQ
WQINKDVLAVIEEVIRLDLGYGVPSFKPLIDKENKPANPVPVEFQHLRGR
ELKEMLSPEQWQQFINWKGECARLYTAETKRGSKSAAVVRMVGQARKYSA
FESIYFVYAMDSRSRVYVQSSTLSPQSNDLGKALLRFTEGRPVNGVEALK
WFCINGANLWGWDKKTFDVRVSNVLDEEFQDMCRDIAADPLTFTQWAKAD
APYEFLAWCFEYAQYLDLVDEGRADEFRTHLPVHQDGSCSGIQHYSAMLR
DEVGAKAVNLKPSDAPQDIYGAVAQVVIKKNALYMDADDATTFTSGSVTL
SGTELRAMASAWDSIGITRSLTKKPVMTLPYGSTRLTCRESVIDYIVDLE
EKEAQKAVAEGRTANKVHPFEDDRQDYLTPGAAYNYMTALIWPSISEVVK
APIVAMKMIRQLARFAAKRNEGLMYTLPTGFILEQKIMATEMLRVRTCLM
GDIKMSLQVETDIVDEAAMMGAAAPNFVHGHDASHLILTVCELVDKGVTS
IAVIHDSFGTHADNTLTLRVALKGQMVAMYIDGNALQKLLEEHEVRWMVD
TGIEVPEQGEFDLNEIMDSEYVFA

A suitable SP6 RNA polymerase can be any enzyme having substantially the same polymerase activity as bacteriophage SP6 RNA polymerase. Thus, in some embodiments, an SP6 RNA polymerase suitable for the present invention may be modified from SEQ ID NO: 26. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 835, 82%, 81%, 80%, 75%, 70%, 65%, or 60% identical or homologous to SEQ ID NO: 26. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

In some embodiments, an SP6 RNA polymerase is encoded by a gene having the following nucleotide sequence:

(SEQ ID NO: 27)
ATGCAAGATTTACACGCTATCCAGCTTCAATTAGAAGAAGAGATGTTTAA
TGGTGGCATTCGTCGCTTCGAAGCAGATCAACAACGCCAGATTGCAGCAG
GTAGCGAGAGCGACACAGCATGGAACCGCCGCCTGTTGTCAGAACTTATT
GCACCTATGGCTGAAGGCATTCAGGCTTATAAAGAAGAGTACGAAGGTAA
GAAAGGTCGTGCACCTCGCGCATTGGCTTTCTTACAATGTGTAGAAAATG
AAGTTGCAGCATACATCACTATGAAAGTTGTTATGGATATGCTGAATACG
GATGCTACCCTTCAGGCTATTGCAATGAGTGTAGCAGAACGCATTGAAGA
CCAAGTGCGCTTTTCTAAGCTAGAAGGTCACGCCGCTAAATACTTTGAGA
AGGTTAAGAAGTCACTCAAGGCTAGCCGTACTAAGTCATATCGTCACGCT
CATAACGTAGCTGTAGTTGCTGAAAAATCAGTTGCAGAAAAGGACGCGGA
CTTTGACCGTTGGGAGGCGTGGCCAAAAGAAACTCAATTGCAGATTGGTA
CTACCTTGCTTGAAATCTTAGAAGGTAGCGTTTTCTATAATGGTGAACCT
GTATTTATGCGTGCTATGCGCACTTATGGCGGAAAGACTATTTACTACTT
ACAAACTTCTGAAAGTGTAGGCCAGTGGATTAGCGCATTCAAAGAGCACG
TAGCGCAATTAAGCCCAGCTTATGCCCCTTGCGTAATCCCTCCTCGTCCT
TGGAGAACTCCATTTAATGGAGGGTTCCATACTGAGAAGGTAGCTAGCCG
TATCCGTCTTGTAAAAGGTAACCGTGAGCATGTACGCAAGTTGACTCAAA
AGCAAATGCCAAAGGTTTATAAGGCTATCAACGCATTACAAAATACACAA
TGGCAAATCAACAAGGATGTATTAGCAGTTATTGAAGAAGTAATCCGCTT
AGACCTTGGTTATGGTGTACCTTCCTTCAAGCCACTGATTGACAAGGAGA
ACAAGCCAGCTAACCCGGTACCTGTTGAATTCCAACACCTGCGCGGTCGT
GAACTGAAAGAGATGCTATCACCTGAGCAGTGGCAACAATTCATTAACTG
GAAAGGCGAATGCGCGCGCCTATATACCGCAGAAACTAAGCGCGGTTCAA
AGTCCGCCGCCGTTGTTCGCATGGTAGGACAGGCCCGTAAATATAGCGCC
TTTGAATCCATTTACTTCGTGTACGCAATGGATAGCCGCAGCCGTGTCTA
TGTGCAATCTAGCACGCTCTCTCCGCAGTCTAACGACTTAGGTAAGGCAT
TACTCCGCTTTACCGAGGGACGCCCTGTGAATGGCGTAGAAGCGCTTAAA
TGGTTCTGCATCAATGGTGCTAACCTTTGGGGATGGGACAAGAAAACTTT
TGATGTGCGCGTGTCTAACGTATTAGATGAGGAATTCCAAGATATGTGTC
GAGACATCGCCGCAGACCCTCTCACATTCACCCAATGGGCTAAAGCTGAT
GCACCTTATGAATTCCTCGCTTGGTGCTTTGAGTATGCTCAATACCTTGA
TTTGGTGGATGAAGGAAGGGCCGACGAATTCCGCACTCACCTACCAGTAC
ATCAGGACGGGTCTTGTTCAGGCATTCAGCACTATAGTGCTATGCTTCGC
GACGAAGTAGGGGCCAAAGCTGTTAACCTGAAACCCTCCGATGCACCGCA
GGATATCTATGGGGCGGTGGCGCAAGTGGTTATCAAGAAGAATGCGCTAT
ATATGGATGCGGACGATGCAACCACGTTTACTTCTGGTAGCGTCACGCTG
TCCGGTACAGAACTGCGAGCAATGGCTAGCGCATGGGATAGTATTGGTAT
TACCCGTAGCTTAACCAAAAAGCCCGTGATGACCTTGCCATATGGTTCTA
CTCGCTTAACTTGCCGTGAATCTGTGATTGATTACATCGTAGACTTAGAG
GAAAAAGAGGCGCAGAAGGCAGTAGCAGAAGGGCGGACGGCAAACAAGGT
ACATCCTTTTGAAGACGATCGTCAAGATTACTTGACTCCGGGCGCAGCTT
ACAACTACATGACGGCACTAATCTGGCCTTCTATTTCTGAAGTAGTTAAG
GCACCGATAGTAGCTATGAAGATGATACGCCAGCTTGCACGCTTTGCAGC
GAAACGTAATGAAGGCCTGATGTACACCCTGCCTACTGGCTTCATCTTAG
AACAGAAGATCATGGCAACCGAGATGCTACGCGTGCGTACCTGTCTGATG
GGTGATATCAAGATGTCCCTTCAGGTTGAAACGGATATCGTAGATGAAGC
CGCTATGATGGGAGCAGCAGCACCTAATTTCGTACACGGTCATGACGCAA
GTCACCTTATCCTTACCGTATGTGAATTGGTAGACAAGGGCGTAACTAGT
ATCGCTGTAATCCACGACTCTTTTGGTACTCATGCAGACAACACCCTCAC
TCTTAGAGTGGCACTTAAAGGGCAGATGGTTGCAATGTATATTGATGGTA
ATGCGCTTCAGAAACTACTGGAGGAGCATGAAGTGCGCTGGATGGTTGAT
ACAGGTATCGAAGTACCTGAGCAAGGGGAGTTCGACCTTAACGAAATCAT
GGATTCTGAATACGTATTTGCCTAA.

A suitable gene encoding the SP6 RNA polymerase may be about 99%, 98%, 97%, 96%, 95%, 94%, 93%, 92%, 91%, 90%, 89%, 88%, 87%, 86%, 85%, 84%, 83%, 82%, 81%, or 80% identical or homologous to SEQ ID NO: 27.

A suitable SP6 RNA polymerase may be a commercially available product, e.g., from Ambion, New England Biolabs (NEB), Promega, and Roche. The SP6 may be ordered and/or custom designed from a commercial source or a non-commercial source according to the amino acid sequence of SEQ ID NO: 26 or a variant of SEQ ID NO: 26 as described herein. The SP6 RNA polymerase may be a standard-fidelity polymerase or may be a high-fidelity/high-efficiency/high-capacity polymerase which has been modified to promote RNA polymerase activities, e.g., mutations in the SP6 RNA polymerase gene or post-translational modifications of the SP6 RNA polymerase itself. Examples of such modified SP6 RNA polymerase include SP6 RNA Polymerase-Plus™ from Ambion, HiScribe SP6 from NEB, and RiboMAX™ and Riboprobe® Systems from Promega.

In some embodiments, the SP6 RNA polymerase is thermostable. In a particular embodiment, the amino acid sequence of an SP6 RNA polymerase for use with the invention contains one or more mutations relative to a wild-type SP6 polymerase that render the enzyme active at temperatures ranging from 37° C. to 56° C. In some embodiments, an SP6 RNA polymerase for use with the invention functions at an optimal temperature of 50° C.-52° C. In other embodiments, an SP6 RNA polymerase for use with the invention has a half-life of at least 60 minutes at 50° C. For example, a particularly suitable SP6 RNA polymerase for use with the invention has a half-life of 60 minutes to 120 minutes (e.g., 70 minutes to 100 minutes, or 80 minutes to 90 minutes) at 50° C.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., in Costa et al. Frontiers in Microbiology 5 (2014): 63 and in PCT/US16/57044, the contents of which are incorporated herein by reference in their entireties. In some embodiments, a His tag is located at SP6's N-terminus.

### T7 RNA Polymerase

In some embodiments, the mRNA is synthesized by a T7 RNA polymerase.

T7 RNA polymerase is a DNA-dependent RNA polymerase with high sequence specificity for T7 promoter sequences. Typically, T7 RNA polymerase catalyzes the 5′→3′ in vitro synthesis of RNA on either single-stranded DNA or double-stranded DNA downstream from its promoter. T7 RNA polymerase incorporates native ribonucleotides and/or modified ribonucleotides into the polymerized transcript.

In some embodiments, the T7 RNA polymerase is thermostable. In a particular embodiment, the amino acid sequence of a T7 RNA polymerase for use with the invention contains one or more mutations relative to a wild-type T7 polymerase that render the enzyme active at temperatures ranging from 37° C. to 56° C. An example for a suitable RNA polymerase is Hi-T7® RNA Polymerase from NEB. In some embodiments, a T7 RNA polymerase for use with the invention functions at an optimal temperature of 50° C.-52° C. In other embodiments, a T7 RNA polymerase for use with the invention has a half-life of at least 60 minutes at 50° C. For example, a particularly suitable T7 RNA polymerase for use with the invention has a half-life of 60 minutes to 120 minutes (e.g., 70 minutes to 100 minutes, or 80 minutes to 90 minutes) at 50° C.

### Nucleotides

Various naturally occurring or modified nucleosides may be used to produce an mRNA transcript according to the present invention. In a typical embodiment, an mRNA transcript is synthesized with natural nucleosides (i.e., adenosine, guanosine, cytidine, and uridine). In other embodiments, an mRNA transcript is synthesized with natural nucleosides (e.g., adenosine, guanosine, cytidine, and uridine) and one or of the following: nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”) (see, e.g., U.S. Pat. No. 8,278,036 or WO2011012316 for a discussion of such residues and their incorporation into mRNA). The mRNA may be RNA, which is defined as RNA in which 25% of U residues are 2-thio-uridine and 25% of C residues are 5-methylcytidine. Teachings for the use of RNA are disclosed in US Patent Publication US20120195936 and international publication WO2011012316, each of which is hereby incorporated by reference in its entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but is not limited to, a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides, for example, more than 0%, 1%, 10%, 25%, 50%, 75%, 85%, 90%, 95%, or 100% of the constituent nucleotides may be modified individually or in combination.

### Reaction Conditions

The reaction mixture may be incubated at about 37° C. to about 56° C. for thirty minutes to six hours, e.g., about sixty to about ninety minutes. In some embodiments, incubation takes place at about 37° C. to about 42° C. In other embodiments, incubation takes place at about 43° C. to about 56° C., e.g., at about 50° C. to about 52° C. In certain embodiments, the yield of accurately terminated mRNA transcripts obtained in an in vitro transcription reaction is increased significantly by including two or more termination signals in the plasmid which encodes a nucleotide sequence to be transcribed into an mRNA transcript of interest and performing the in vitro transcription reaction at a temperature of about 50° C. to about 52° C.

In some embodiments, about 5 mM NTPs, about 0.05 mg/mL RNA polymerase, and about 0.1 mg/ml plasmid in a suitable RNA polymerase reaction buffer (final reaction mixture pH of about 7.5) are incubated at about 37° C. to about 42° C. for 60 minutes to 180 minutes. In other embodiments, about 5 mM NTPs, about 0.05 mg/ml RNA polymerase, and about 0.1 mg/ml plasmid in a suitable RNA polymerase reaction buffer (final reaction mixture pH of about 7.5) are incubated at about 50° C. to about 52° C. for sixty to ninety minutes.

In a particular embodiment, the in vitro transcription reaction mixture comprises about 0.1 mg/ml RNA polymerase (e.g., SP6 RNA polymerase), and about 0.05 mg/ml or more (e.g., 0.07 mg/ml or more) plasmid in a suitable RNA polymerase reaction buffer. The inventors have found that optimizing the amounts of plasmid and RNA polymerase in the in vitro transcription reaction mixture relative to standard conditions can improve mRNA transcript yield in high-throughput screening methods of the invention.

In some embodiments, a reaction mixture contains the plasmid, RNA polymerase, RNase inhibitor, pyrophosphatase, 29 mM NTPs, 10 mM DTT and a reaction buffer (when at 10× the reaction buffer is 800 mM HEPES, 20 mM spermidine, 250 mM MgCl₂, pH 7.7) and quantity sufficient (QS) to a desired reaction volume with RNase-free water. This reaction mixture may then be incubated at 37° C. for about 90-120 minutes. Subsequently, the plasmid may be removed by incubating the reaction mixture in the presence of a DNase. An incubation time of about 15 minutes is typically sufficient. For example, the in vitro transcription reaction can be quenched by addition of DNase I and a DNase I buffer (when at 10× the DNase I buffer is 100 mM Tris-HCl, 5 mM MgCl₂ and 25 mM CaCl₂), pH 7.6) to facilitate digestion of the plasmid in preparation for purification of the resulting mRNA transcript.

In some embodiments, a reaction mixture includes NTPs at a concentration ranging from 1-10 mM, the plasmid at a concentration ranging from 0.05-0.2 mg/ml, and RNA polymerase (e.g., an SP6 RNA polymerase) at a concentration ranging from 0.01-0.1 mg/ml. In particular embodiments, the reaction mixture comprises NTPs at a concentration of about 5 mM, the plasmid at a final concentration of about 0.06 mg/ml to about 0.12 mg/ml, and the RNA polymerase at a concentration of about 0.05 mg/ml to about 0.1 mg/ml. In a specific embodiment, the reaction mixture comprises NTPs at a concentration of about 5 mM, the plasmid at a concentration of about 0.07 mg/ml, and the RNA polymerase at a concentration of about 0.1 mg/ml.

### Post-Synthesis Processing

In some embodiments, the method of the present invention further comprises a separate step of capping and/or tailing the in vitro transcribed mRNA transcripts.

For example, both a 5′ cap and a 3′ tail may be added after the synthesis of an mRNA transcript composed of a 5′ UTR, a nucleotide sequence of interest, and a 3′ UTR. The presence of the cap is important in providing resistance to nucleases found in most eukaryotic cells and is typically required to achieve high levels of expression from an mRNA transcript. The presence of a “tail” similarly serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp(5′)(2′OMeG), m7G(5′)ppp(5′)(2′OMeA), m7(3′OMeG)(5′)ppp(5′)(2′OMeG), m7(3′OMeG)(5′)ppp(5′)(2′OMeA), m7G(5′)ppp (5′(A,G(5′)ppp(5′)A and G(5′)ppp(5′)G. In a specific embodiment, the cap structure is m7G(5′)ppp(5′)(2′OMeG). Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly(A) or poly(C) tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly(A) or poly(C) tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides), respectively. In some embodiments, a tail structure includes a combination of poly(A) and poly(C) tails with various lengths as described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, %%, 97%, 98%, or 99% cytosine nucleotides.

In some embodiments of a screening method of the invention, a poly(A) tail is added post-transcriptionally by incubating an mRNA transcript in the presence of a poly(A) polymerase. In a particular embodiment, the amount of poly(A) polymerase is adjusted to account for the length of the mRNA transcript. For example, mRNA transcripts with a length of 0.1 to 1 kilobases (kb) are incubated with a given amount of poly(A) polymerase (e.g., the amount recommended by the manufacturer of the poly(A) polymerase). This amount is halved for mRNA transcripts with a length of 1 kb to 2 kb (to 2× of the given amount), halved again for mRNA transcripts with a length of 2 kb to 4 kb (to 4× of the given amount), and halved again for mRNA transcripts with a length of 4 kb to 8 kb (to 8× of the given amount). In a particular embodiment, each full-length mRNA transcript comprises a poly(A) tail that is 60 nucleotides to 200 nucleotides long.

### Screening of Nucleotide Sequences of Interest for High Protein Expression

Steps (e) and (f) of a screening method of the invention identify nucleotide sequences encoding a protein generated by a codon optimization algorithm that are transcribed into full-length mRNA transcripts. In some embodiments, it may be desirable to further narrow the pool of candidate nucleotide sequences to those that yield the largest amount of the encoded protein. Accordingly, in some embodiments, a screening method in accordance with the invention can further comprise the following steps:
g. for each nucleotide sequence selected in step (f), transfecting a cell with the full-length mRNA transcript;
h. for each cell transfected in step (g), determining the amount of the encoded protein expressed from the full-length mRNA transcript; and
i. selecting the nucleotide sequence, whose full-length mRNA transcript yields the largest amount of the encoded protein.

The cell may be cultured in vitro. In some embodiments, the cell may form part of an organism, e.g., a suitable experimental animal such as a mouse, a rat, or a non-human primate (in vivo screening). Suitable transfection agents for in vitro and in vivo transfection of cells may be used. For example, a suitable transfection agent for in vitro applications is Lipofectamine. For in vivo transfection of cells in an experimental animal, the full-length mRNA transcript may be encapsulated in a lipid nanoparticle. Suitable lipid nanoparticles typically comprise a cationic lipid, a non-cationic lipid, a PEG-modified lipid, and optionally cholesterol or a cholesterol derivative.

Methods for determining the amount of the encoded protein expressed from the full-length mRNA transcript are known. Such methods include SDS PAGE, and optionally western blotting, to determine expression of the encoded protein. In some embodiments, the functional activity of the protein encoded by the full-length mRNA transcript is determined. These methods may vary depending on the properties of the encoded protein of interest. For example, wherein the encoded protein is an enzyme, an enzyme activity assay may be used to determine the functional enzymatic activity of the protein encoded by the full-length mRNA transcript.

### Purification

### DNA Purification

A screening method of the invention may further comprise one or more purification steps. For example, depending on the method used to provide the vector backbone in step (c) or the assembly of the plasmid in step (d) of the screening method of the invention, one or more purification steps may be required to remove enzyme reagents and/or buffers used in steps (c) and/or step (d) before proceeding to the in vitro transcription step (e). In a specific embodiment, the one or more purification steps comprises purifying the plasmid to remove one or more enzyme(s) used for assembly in step (d).

In some embodiments, the plasmid generated in step (d) of a screening method of the invention is transformed into a bacterial cell, typically an Escherichia coli cell, in order to amplify the assembled plasmid. Any suitable transformation method may be used, including chemical transformation or electroporation. Chemically competent or electrocompetent bacterial cells are readily available from commercial suppliers. Transformed cells are typically grown on a suitable growth medium. In order to perform the in vitro transcription in step (e), the plasmid is typically extracted from the bacterial cell and purified. The plasmid may be extracted directly from a bacterial colony grown on a solid growth medium. Alternatively, in order to obtain larger quantities of the plasmid, the plasmid may be extracted from a liquid bacterial culture. For example, a liquid bacterial culture (e.g., an Escherichia coli culture) may be grown under suitable conditions in an appropriate growth medium (e.g., Terrific Broth or Luria Broth). Cultures can be grown in a format suitable for high-throughput screening methods (e.g., in 96-well plates). A commonly used extraction method to release a plasmid from bacterial cells is alkaline lysis. Alkaline lysis involves the addition of an alkaline solution (e.g., 200 mM NaOH), typically in the presence of a detergent (e.g., Triton X-100, Tween, SDS or combinations thereof, at a suitable concentration, e.g., about 1%). Purification of the extracted plasmid (e.g., from a bacterial lysate) can be done with the same method that is used to remove enzyme reagents and/or buffers used in step (c) of the screening method.

In a particular embodiment, purification comprises precipitating a plasmid by adding (i) a chaotropic salt, and (ii) an alcohol and/or an amphiphilic polymer to an impure preparation comprising the plasmid. In some embodiments, the chaotropic salt (e.g., a guanidinium salt) is at a final concentration of about 0.1 M to about 4 M. For example, in some embodiments, the chaotropic salt (e.g., a guanidinium salt) may be at a final concentration of about 0.1 M to about 0.4 M, e.g., about 125 mM, about 250 mM or about 375 mM. A final concentration of the chaotropic salt of 0.1 M to 0.4 M may be particularly suitable for purifying supercoiled plasmid DNA. In other embodiments, the chaotropic salt (e.g., a guanidinium salt) is at a final concentration of about 1.5 M to about 2.7 M, e.g., about 1.9 M or 2.5 M. A final concentration of the chaotropic salt of about 1.5 M to about 2.7 M may be particularly suitable for precipitating linear nucleic acids, such as a linearized plasmid DNA. In particular embodiments, the chaotropic salt is a guanidinium salt, e.g., guanidinium thiocyanate (GSCN). In some embodiments, the amphiphilic polymer is selected from pluronics, polyvinyl pyrrolidone, polyvinyl alcohol, polyethylene glycol (PEG), triethylene glycol monomethyl ether (MTEG), or combinations thereof. In some embodiments, the amphiphilic polymer is MTEG. In some embodiments, the alcohol is isopropanol or ethanol. In some embodiments, purification of a plasmid by precipitation comprises adding both an amphiphilic polymer (e.g., MTEG) and an alcohol (e.g., isopropanol) to an impure preparation comprising the plasmid. More typically, an amphiphilic polymer (e.g., MTEG) is used for precipitation together with a chaotropic salt (e.g., a guanidinium salt).

In particular embodiments, purification comprises precipitating the plasmid by adding a chaotropic guanidinium salt such as GSCN, and an amphiphilic polymer such as MTEG or PEG (e.g., PEG having a molecular weight of about 4000 to about 8000 g/mol). In a specific embodiment, purification comprises precipitating the plasmid by adding 0.1-0.3 volumes of the chaotropic guanidinium salt at a high molar concentration (e.g., ≥4 M, for instance 5 M GSCN) and 1-3 volumes 100% MTEG (v/v) solution to 1 volume of plasmid. The precipitation conditions may be adjusted appropriately for specific applications. For example, purification of linearized plasmid may be achieved by adding 0.1-0.3 volumes of the chaotropic guanidinium salt at a concentration of 4-6 M (e.g., 5 M GSCN) and 1-3 volumes 100% MTEG (v/v) to 1 volume of linearized plasmid. In a specific embodiment, purification of linearized plasmid is achieved by adding 0.1 volumes of 5 M GSCN and 2 volumes 100% MTEG (v/v) to 1 volume of linearized plasmid. Purification of supercoiled plasmid from filtered lysates of a bacterial culture may be achieved by adding 0.1 volume of 5 M GSCN and 1.35 volumes 100% MTEG (v/v) to 1 volume of lysate. In some embodiments, 100% MTEG may be replaced with 100% ethanol. In some embodiments, 100% MTEG may be replaced with 100% isopropanol.

In some embodiments, a combination of an amphiphilic polymer and an alcohol is used to improve the recovery of plasmid DNA after purification. For example, MTEG and isopropanol may be used in combination during purification. In particular embodiments, purification of linearized plasmid is achieved by adding a guanidinium salt at a final concentration of 2-3 M, MTEG at a final concentration of about 30-40% MTEG (v/v) and at a final concentration of about 10-20% isopropanol to an impure preparation comprising the linearized plasmid (typically about 10-30 μg total plasmid DNA). In a specific embodiment, purification of linearized plasmid is achieved by adding 0.1 volumes of 5 M GSCN and 2 volumes 100% MTEG (v/v) to 1 volume of linearized plasmid, followed by the addition of 3 volumes of a buffer comprising 5 M guanidine hydrochloride and 30% isopropanol. The inventors have found that these conditions are particularly suitable to obtain a high yield of purified plasmid DNA when magnetic particles (e.g., silica-coated beads) are used for the subsequent steps of purification.

In some embodiments, the precipitated plasmid DNA may be mixed with a buffered solution. The buffered solution may have a pH of about 5 to about 6 (e.g., 5.5). In some embodiments, the buffered solution comprises an acid (e.g., potassium acetate), typically at a concentration suitable for neutralizing a lysate obtained by alkaline lysis. In a specific embodiment, the buffered solution is added in such an amount that the final potassium acetate concentration is about 1 M to about 2 M.

The precipitated plasmid DNA can be washed by passing the resulting suspension through a filter plate that retains the precipitated plasmid DNA. A filter (e.g., made of a glass fibers) with an average pore size of about 0.7 μm or less (e.g., from 0.7 μm to 0.5 μm) has been found to be suitable for retaining precipitated plasmid DNA. The retentate can then be washed with a suitable washing solution that maintains the plasmid DNA in precipitated form. A suitable wash solution comprises an alcohol (e.g., ethanol or isopropanol) or an amphiphilic polymer (e.g., MTEG) in an amount sufficient to maintain the plasmid DNA in precipitated form. For example, a suitable wash solution comprises the alcohol (e.g., ethanol) at a 50%, 60%, 70%, 80%, 90% or more volume/volume (v/v) concentration. In a particular embodiment, the wash solution comprises an alcohol (e.g., ethanol) at about 70% to 80% (v/v) concentration (e.g., 70% (v/v) ethanol or 80% (v/v) ethanol). In another particular embodiment, a suitable wash solution is 100% isopropanol. The washed precipitated plasmid DNA can be eluted from the filer with sterile water (which is typically RNase-free).

In a particular embodiment, the precipitated plasmid DNA is bound to a DNA-binding magnetic particle. Suitable DNA-binding magnetic particles include silica-coated beads with a metallic core (e.g., iron, nickel or cobalt, typically iron). The magnetic particle with the bound precipitate is then transferred one or more times to one or more receptacles with a wash solution. A suitable wash solution comprises an alcohol (e.g., isopropanol or ethanol) at a 50%, 60%, 70%, 80%, 90% or more (v/v) concentration. In a particular embodiment, the wash solution comprises an alcohol (e.g., ethanol) at about 70% to 80% (v/v) concentration (e.g., 70% (v/v) ethanol or 80% (v/v) ethanol). In another particular embodiment, a suitable wash solution is 100% isopropanol.

The inventors have found that precipitating the plasmid by adding a chaotropic guanidinium salt such as GSCN and an amphiphilic polymer such as MTEG and washing the precipitate on a filter with a suitable wash solution that maintains the plasmid in precipitated form results in higher yields of purified plasmid DNA. Yield and purity can be assessed by suitable methods such as gel electrophoresis or capillary electrophoresis.

In some embodiments, a plasmid for use with a screening method of the invention is purified by high-throughput purification method as described herein. For example, a plasmid for use with a screening method of the invention may be purified by (i) adding a chaotropic guanidinium salt (such as GSCN) in combination with an amphiphilic polymer (such as MTEG) and/or an alcohol, to an impure plasmid preparation to form a precipitate comprising the plasmid, (ii) binding the precipitate to a DNA-binding magnetic particle (e.g., a silica-coated bead with a metallic core), (iii) washing the precipitate on the DNA-binding magnetic particle one or more times with a suitable wash solution (e.g., 70% ethanol), and (iv) eluting the precipitate from the DNA-binding magnetic bead in a suitable elution medium such as sterile water. These purification steps can be performed by an automated liquid handling system (typically utilizing a commonly used multi-well format such as 96-well plates) so that a plurality of plasmids can be purified in parallel. The inventors have found that the use of an automated liquid handling system in the purification of the plasmid DNA in a screening method of the invention is particularly advantageous because it reduces the processing time dramatically while increasing the plasmid yield at the same time.

When DNA-binding magnetic particles are used to purify plasmid from an impure preparation, the precipitate comprising the plasmid may be formed under conditions that favor the binding of the precipitate to the DNA-binding magnet particles (e.g., a silica-coated bead with a metallic core). In particular embodiments, such conditions are achieved by adding a guanidinium salt at a final concentration of 2-3 M, MTEG at a final concentration of about 30-40% MTEG (v/v) and isopropanol at a final concentration of about 10-20% to an impure preparation comprising the plasmid (typically about 10-30 μg total plasmid DNA). In a specific embodiment, purification of plasmid is achieved by adding 0.1 volumes of 5 M GSCN and 2 volumes 100% MTEG (v/v) to 1 volume of plasmid, followed by the addition of 3 volumes of a binding solution comprising 5 M guanidine hydrochloride and 30% isopropanol. In some embodiments, the plasmid is linearized. Accordingly, in some embodiments, the impure preparation is a restriction digest comprising linearized plasmid. The inventors have found that these conditions are particularly suitable to obtain a high yield of purified plasmid DNA when magnetic particles (e.g., silica-coated beads) are used during high-throughput purification.

### RNA Purification

A screening method of the invention may further comprise one or more steps of purifying mRNA transcripts obtained in step (e) from enzyme reagents and other components of the in vitro transcription reaction mixture. Such purification steps may also be used to remove enzyme reagents and other components used in the post-synthesis processing of the mRNA transcripts (e.g., to remove enzymes used in capping and/or tailing reactions).

In a particular embodiment, purification comprises precipitating mRNA transcripts by adding to an impure preparation comprising the mRNA transcripts (i) a chaotropic salt (e.g., a guanidinium salt), and (ii) an alcohol or an amphiphilic polymer. In some embodiments, the chaotropic salt (e.g., a guanidinium salt) is at a final concentration of about 2 M to about 3 M, for example, about 2.0 M to about 2.5 M (e.g., about 2.3 M). In a specific embodiment, the chaotropic salt is guanidinium thiocyanate (GSCN). In some embodiments, the amphiphilic polymer is selected from pluronics, polyvinyl pyrrolidone, polyvinyl alcohol, polyethylene glycol (PEG), triethylene glycol monomethyl ether (MTEG), or combinations thereof. In a specific embodiment, the amphiphilic polymer is MTEG. In another embodiment, the alcohol is isopropanol or ethanol.

In a particular embodiment, the mRNA transcripts are precipitated by adding to an impure preparation comprising the mRNA transcripts (i) a chaotropic salt (such as a guanidinium salt, e.g., GSCN) and (ii) an alcohol (e.g., isopropanol or ethanol). In some embodiments, 1 volume of an impure preparation comprising the mRNA transcripts is added to 2-3 volumes of the chaotropic salt (e.g., a guanidinium salt such as GSCN at a high molar concentration, e.g., ≥4 M) and 1.5-2 volumes of the alcohol. In a specific embodiment, 1 volume of an impure preparation comprising the mRNA transcripts is added to 2.3 volumes of GSCN (5 M; final concentration: about 2.3 M) and 1.7 volumes of absolute isopropanol (final concentration: about 34%) to precipitate the mRNA transcripts from the impure preparation to form an mRNA precipitate.

The resulting mRNA precipitate is then captured on a suitable filter medium. A suitable filter medium may have a pore size of about 0.7 μm to about 1.2 μm pores. A suitable material may be glass (e.g., a glass filter). The captured mRNA precipitate is then washed with a suitable wash solution, typically comprising an alcohol (e.g., 100% isopropanol or 80% (v/v) ethanol). In some embodiments, the wash solution is pushed through the filter medium by applying positive pressure. The mRNA precipitate can be solubilized in RNase-free water to provide purified mRNA transcripts.

The inventors have found that using precipitation-based mRNA purification methods is advantageous because they can be easily incorporated into a high-throughput process and provide a high yield of purified mRNA transcripts (≥120 μg per sample), even when a 96-well plate format is used. Moreover, the clogging of the filter medium (e.g., due to RNA “gel” formation on the filter) can typically be avoided.

### EXAMPLES

The following examples are included for illustrative purposes only and are not intended to limit the scope of the invention.

### Example 1. Screening Optimized Nucleotide Sequences for Use in Therapeutic mRNAs

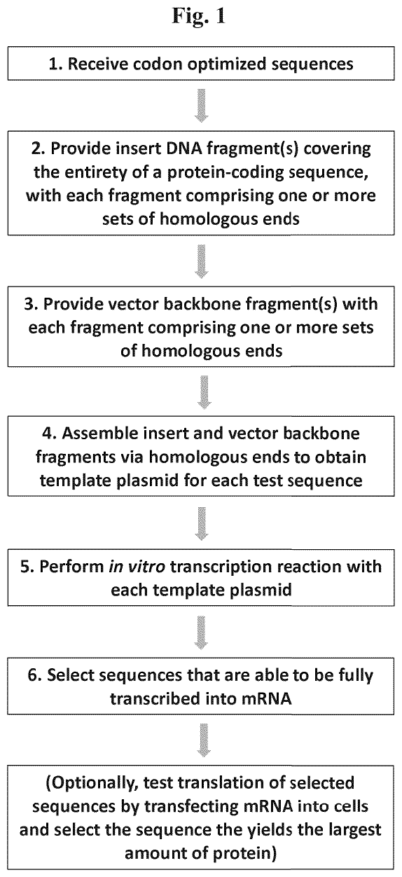

This example outlines the overall scheme of the screening methods according to the invention. FIG. 1 provides a schematic illustration. As described above, these methods are particularly useful for the screening of large numbers of protein-coding nucleotide sequences to be used in therapeutic mRNAs, as they require less time, involve fewer steps and are more easily adaptable to a multi-well plate format.

In a first step, a plurality of nucleotide sequences to be screened are provided by a codon-optimization algorithm. Such a codon-optimization algorithm typically outputs several optimized coding sequences for a protein of interest, whose performance during in vitro transcription (IVT) of an mRNA and its use as a potential therapeutic in vivo needs to be assessed empirically. A codon-optimization algorithm that may be used in accordance with the invention is described in Example 2.

In a second step, for each nucleotide sequence, a set of DNA fragments covering the entirety of the nucleotide sequence is provided. This step may be accomplished using suitable chemical synthesis processes. Currently available chemical synthesis processes are typically more error-prone for longer DNA fragments, exceeding 1 kb in length. Many protein-coding nucleotide sequences of therapeutic interest exceed a size of 1 kb. To take advantage of the available chemical synthesis processes, the protein-coding nucleotide sequence is divided into several DNA fragments that can easily be generated by available chemical synthesis processes with very low error rates. The fragments are provided with two sets of homologous ends: one set to facilitate assembly of the fragments to yield an insert comprising the complete nucleotide sequence; and another set to facilitate assembly of this insert into a vector backbone.

For shorter protein-coding nucleotide sequences, it may not be necessary to divide the sequence into DNA fragments. In this case, the nucleotide sequence is provided as an insert with only one set of homologous ends. This set facilitates the assembly of the insert to a vector backbone.

The DNA fragments forming the complete nucleotide sequence may also comprise a 5′ untranslated region (5′ UTR) and/or a 3′ untranslated region (3′ UTR) and may be operationally linked to an RNA polymerase promoter. The inventors have found that inclusion of the 5′ and 3′ UTRs in the insert rather than the vector backbone can facilitate assembly of a plasmid including the nucleotide sequence of interest.

In a third step, a vector backbone, with homologous ends to facilitate the assembly of the insert into the vector backbone, is provided. The vector backbone may be produced by any suitable method known, such as, for example, restriction enzyme digestion or PCR.

The assembly reaction may be more efficient when fragments of similar sizes are provided. Therefore, the vector backbone may be provided as one or more fragments, wherein each of these fragments is of a similar size to the insert DNA fragments to be used. In this case, the vector fragments will comprise a second set of homologous ends to facilitate assembly of the fragments to yield a vector backbone with a first set of homologous ends. The first set of homologous ends facilitates the assembly of the insert into the vector backbone. The vector fragments may be produced by any suitable method, such as, for example, restriction enzyme digestion or PCR.

In a fourth step, the vector backbone and DNA fragment(s) are assembled to generate a plasmid comprising the nucleotide sequence flanked by a 5′ UTR and a 3′ UTR and operationally linked to an RNA polymerase promoter. An assembly method that may be used in accordance with the invention is described in Example 3. Methods for verification of the assembly reaction are also described in Example 3.

The assembly reaction yields the plasmid template for an in vitro transcription (IVT) reaction which is performed in a fifth step. A method for performing the in vitro transcription reaction is described in Example 5. Prior to this step, the plasmid template may be amplified in E. coli and purified. A DNA purification method that may be used for this is described in Example 4.

In a sixth step, nucleotide sequences that generated full-length mRNA transcripts in the IVT reaction are selected. The inventors have found that not all nucleotide sequences that are optimized to yield maximum expression, e.g., by removing rare codons and optimizing the GC content, are effectively transcribed in an IVT reaction. Some nucleotide sequences may terminate prematurely, e.g., due to the presence of terminator sequences in the nucleotide sequences. To the extent that such terminator sequences are known they can be removed during the sequence optimization step, however, not all such sequences are known. Selecting sequences that result in full-length mRNA transcripts is therefore important to provide mRNAs that have high therapeutic potency and can be produced efficiently at a commercial scale as truncated mRNA transcripts do not contribute to therapeutic efficacy and may be difficult to remove by conventional purification methods.

The selected nucleotide sequences may be further tested for their ability to direct high protein expression levels, e.g., in vivo. This may be carried out by transfecting a cell or animal with a selected full-length mRNA transcript and determining the amount of the encoded protein expressed from the full-length mRNA transcript.

### Example 2. Generating Optimized Nucleotide Sequences

This example illustrates a process for generating optimized nucleotide sequences that are then screened in accordance with the method of the invention.

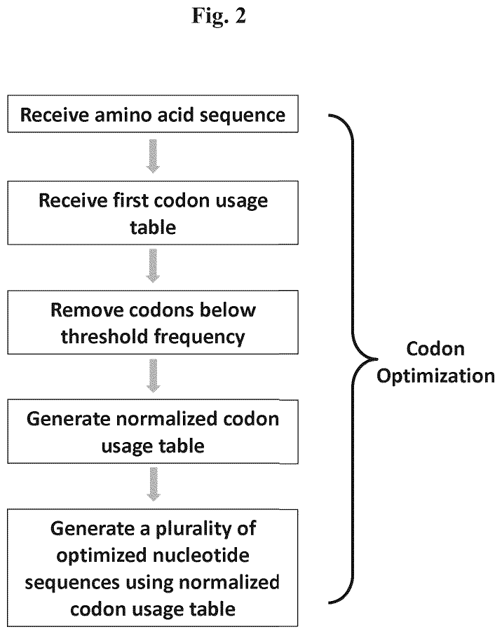

FIG. 2 illustrates a simple codon optimization process. The process receives an amino acid sequence of interest and a first codon usage table which reflects the frequency of each codon in a given organism (namely human codon usage preferences in the context of the present example). The process then removes codons from the first codon usage table if they are associated with a codon usage frequency which is less than a threshold frequency (e.g., 10%). The codon usage frequencies of the codons not removed in the first step are normalized to generate a normalized codon usage table.

Normalizing the codon usage table involves re-distributing the usage frequency value for each removed codon; the usage frequency for a certain removed codon is added to the usage frequencies of the other codons with which the removed codon shares an amino acid. The process uses the normalized codon usage table to generate a list of optimized nucleotide sequences. Each of the codon-optimized nucleotide sequences encode the amino acid sequence of interest.

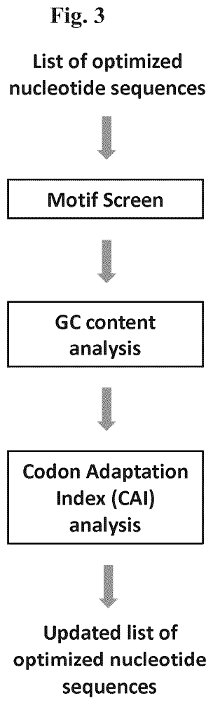

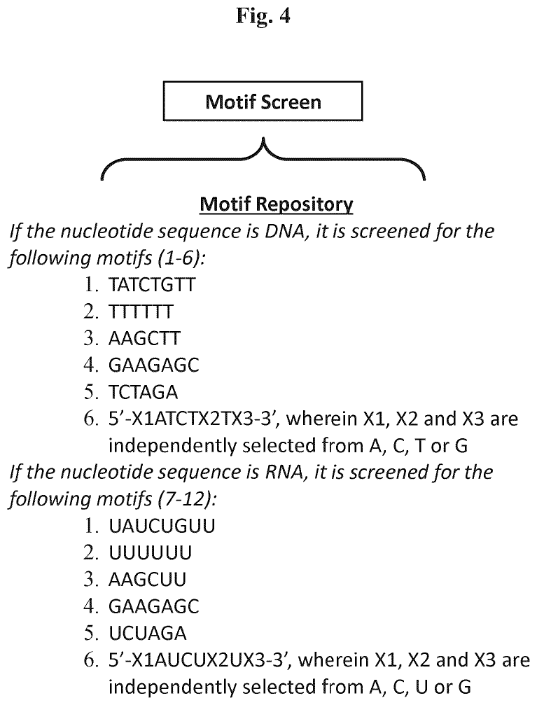

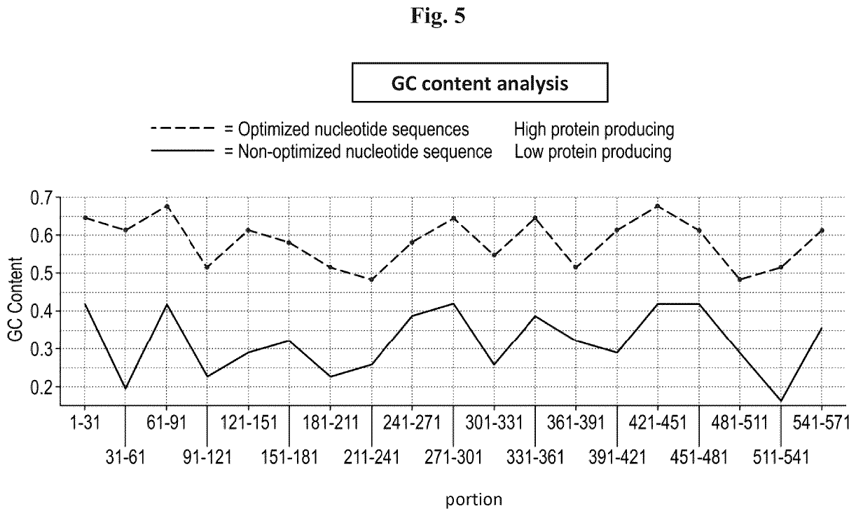

The list of codon-optimized nucleotide sequences is typically further processed as illustrated in FIG. 3. Specifically, a motif screen filter, guanine-cytosine (GC) content analysis filter, and codon adaptation index (CAI) analysis filter are applied to the codon-optimized nucleotide sequences, typically in that order, to generate an updated list of optimized nucleotide sequences. For example, the motif screen filter illustrated in FIG. 4 may be used to remove codon-optimized nucleotide sequences that contain termination sequences that could result in the premature termination of transcription during the IVT reaction. The GC content analysis filter selects codon-optimized nucleotide sequences that have a high GC content across their entire length. As illustrated schematically in FIG. 5, this can be done by dividing the nucleotide sequences into portions of about 30 bases and removing nucleotide sequences in which any portion has a GC content below a certain threshold value.

### Example 3. Rapid Plasmid Assembly

This example describes the assembly of plasmids suitable for adding to an in vitro transcription reaction mixture, using rapid assembly methods in accordance with the screening method of the invention.

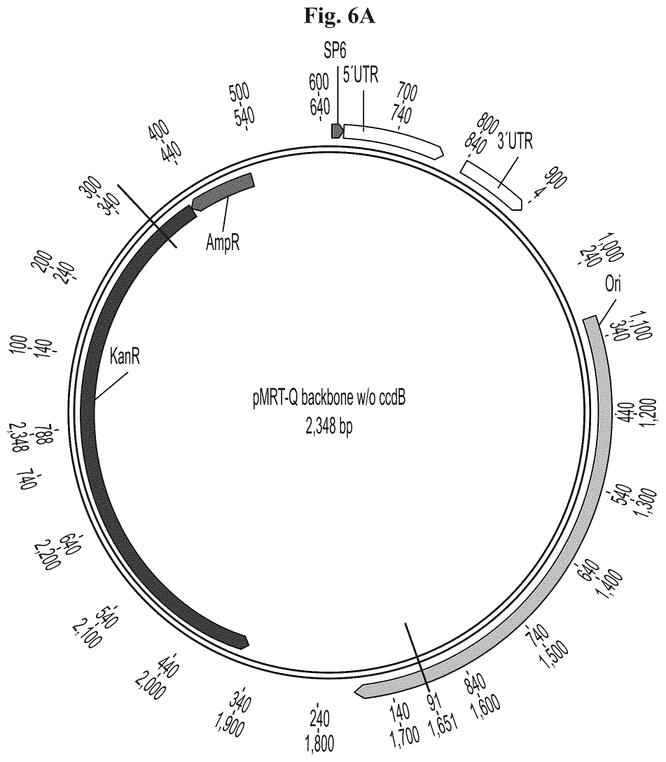

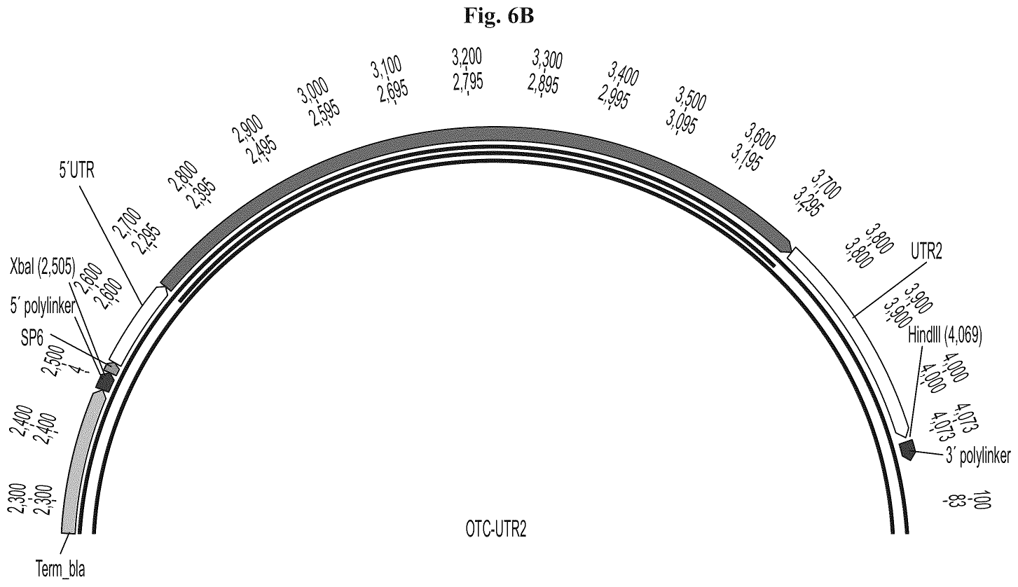

Five optimized nucleotide sequences encoding different proteins of various length were generated using an algorithm as described in Example 2. Each nucleotide sequence was provided as up to four synthesized DNA fragments with homologous ends to facilitate assembly into a plasmid in which the nucleotide sequence was operably linked to a SP6 promoter, 5′ untranslated region (5′ UTR) and 3′ untranslated region (3′ UTR) provided by a vector backbone. The length of each of the optimized nucleotide sequences and the number of DNA fragments for their assembly are given in Table 1. The vector backbone is shown schematically in FIG. 6A. The vector backbone had a length of about 2.4 kb.

TABLE 1

| Test sequence | Length (kb) | Number of fragments |
|---|---|---|
| 1 | 0.8 | 1 |
| 2 | 2.5 | 2 |
| 3 | 3.2 | 3 |
| 4 | 3.7 | 2 |
| 5 | 4 | 4 |

The vector backbone was linearized before use in the assembly reaction. Typically, linearization can be done by restriction enzyme digestion. However, in this example, two PCR-based linearization methods were tested: (i) using one primer set to amplify the vector backbone in one piece, and (ii) using three primer sets to amplify the vector backbone in three pieces (vector fragments) with a set of homologous ends to allow subsequent reassembly. The PCR-amplified vector fragments were isolated by agarose gel electrophoresis and gel extraction using the Monarch® gel extraction kit (New England Biolabs) following the manufacturer's protocol. This scheme is illustrated in FIG. 7.

The vector fragments and DNA fragments were assembled using NEBuilder® HiFi DNA Assembly. For each reaction, about 50 ng of vector backbone was used when the vector backbone was amplified in one piece, and about 50 ng of each piece when the vector backbone was amplified to yield three pieces. The vector backbone and insert fragments were mixed at a vector:insert molar ratio of 1:3 in a volume of ≤10 μl. When the vector backbone was supplied in three pieces, the vector fragments were provided at a 1:1:1 ratio. 10 μl NEBuilder® HiFi DNA Assembly Master Mix was added and the total reaction volume adjusted to 20 μl using deionized water. The assembly reaction was incubated at 50° C. for 1 hour. After completion, Stellar™ chemically competent E. coli cells (Takara Bio) were transformed with 2 μl of the assembly reaction product, according to the manufacturer's instructions.

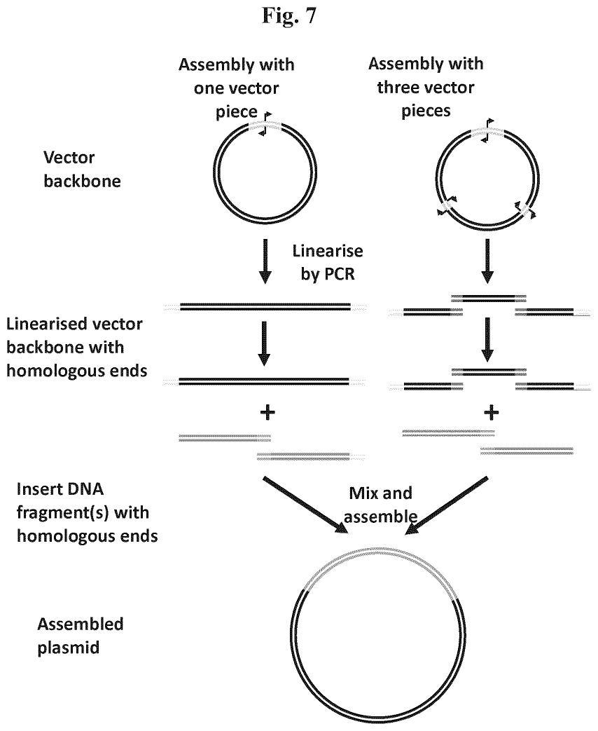

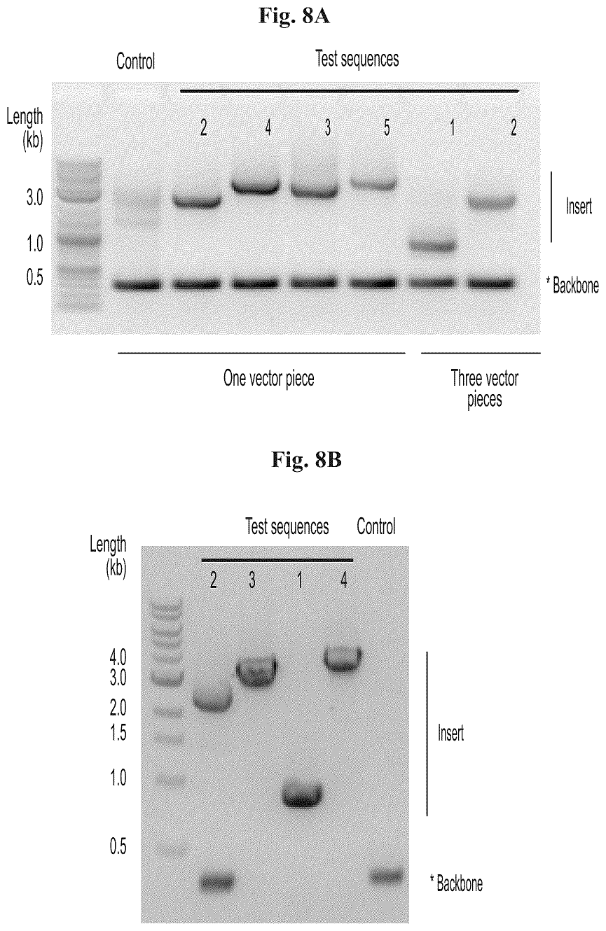

Assembly was verified by (i) PCR of the assembly reaction product and (ii) colony PCR of Escherichia coli transformed with the reaction product, using primers flanking the insert. The binding site of the forward primer comprised the 5′ UTR and SP6 promoter in the vector backbone. The binding site of the reverse primer comprised the 3′ UTR in the vector backbone. The reaction product PCRs (i) were carried out using 1 μl assembly reaction product, 1.25 μl each primer (at 0.5 μM), 12.5 μl Phusion Flash® DNA polymerase master mix, 9 μl deionized water and cycling parameters as recommended by the manufacturer. For the colony PCRs, (ii) colonies were picked into 25 μl sterile water and incubated at 98° C. for 10 minutes. 5 μl of this template was mixed with 1.25 μl of each primer (at 0.5 μM), 12.5 μl Phusion Flash® DNA polymerase master mix and 5 μl deionized water. The cycling parameters were as recommended by the manufacturer. The results of both of these verification PCR methods are shown in FIG. 8A (PCR of the assembly reaction product) and FIG. 8B (colony PCR).

FIG. 8B shows the results of colony PCR performed on Escherichia coli cells transformed with plasmids resulting from assembly reactions in which the vector backbone had been provided as a single piece. In the second lane from the left, the colony contained a mixed population of cells after primary transformation. Part of the cell population contained a plasmid without insert. During colony PCR, the vector backbone was amplified and can be seen at the bottom of the gel.

This example demonstrates rapid assembly methods for plasmids comprising a protein-coding nucleotide sequence which is flanked by a 5′ UTR and a 3′ UTR and operationally linked to an RNA polymerase promoter. As shown in FIG. 8A and FIG. 8B by the presence of an amplified insert band for all of the test reactions, the assembly reactions were successful with a range of vector backbone and insert fragment numbers and sizes. The plasmids produced are suitable for downstream in vitro transcription screening. Unlike traditional genetic cloning techniques, these assembly methods do not require separate restriction enzyme digestion, purification and ligation steps, therefore increasing the speed and scalability of the overall screening method. This makes such methods far more suitable for the high-throughput screening of large numbers of protein-coding nucleotide sequences.

The data also show that assembly of the plasmid can be verified by PCR of the assembly reaction product itself, without the need for transformation into E. coli and colony PCR This allows unsuccessful reactions to be identified on the same day as the assembly reactions are performed, thus helping to prevent potentially time-consuming follow-up of failed reactions.

### Example 4. Precipitation-Based Purification of Plasmid DNA

This example describes the development of a precipitation-based method for the high-throughput purification of plasmid DNA.

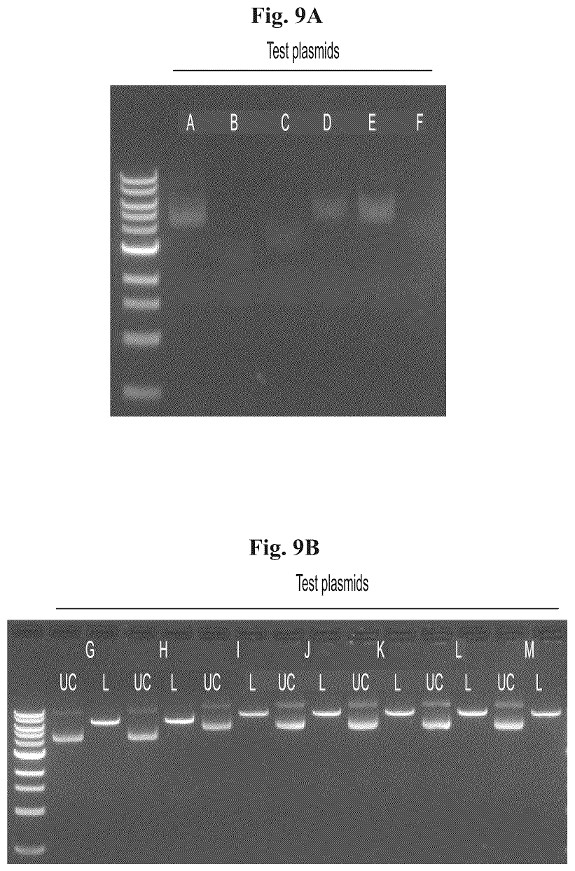

Initially, purification of plasmids obtained in accordance with the methods described in Example 3 was done using the QIAamp® 96 DNA QIAcube® HT Kit according to the manufacturer's protocol. The inventors found, however, that carrying out this standard method for plasmid purification did not produce high yields of DNA. This is demonstrated in FIG. 9A, which shows the result of gel electrophoresis analysis of 6 test plasmids purified according to the QIAGEN® standard protocol; and Table 2, which lists the yield obtained for these plasmids, as quantified by measuring absorbance at 280 nm using a NanoDrop2000 spectrophotometer.

TABLE 2
Low plasmid yields from standard QIAGEN ® purification
protocol

| Test plasmid | Length (kb) | Concentration (ng/μl) |
|---|---|---|
| A | 4.2 | 65.8 |
| B | 3.5 | 74.2 |
| C | 3.8 | 76.4 |
| D | 7.0 | 68.1 |
| E | 6.0 | 73.2 |
| F | 4.6 | 74.5 |

Without wishing to be bound by any particular theory, the inventors reasoned that these low yields may have been due to poor binding of DNA obtained from bacterial lysates. According to the manufacturer's handbook, the QIAamp® 96 DNA QIAcube® HT Kit relies on the selective binding properties of silica-based membranes in the QIAamp 96 plate for DNA purification. To address the problem of poor yield, the inventors investigated an alternative protocol, in which the DNA in the lysate was precipitated using GSCN and MTEG.

In this modified protocol, the steps leading up to the DNA-binding step were the same as in the QIAGEN® protocol. Briefly, for each test plasmid, DH10B Escherichia coli cells (Thermo Scientific™) transformed with the plasmid were used to inoculate terrific broth (TB) supplemented with kanamycin (25-50 μg/ml). The cells were grown overnight at 37° C. in a 96-well plate, with shaking (900 rpm). The cells were recovered by centrifugation and lysed by alkaline lysis using reagents from the QIAamp® 96 DNA QIAcube® HT Kit (QIAGEN®) following the manufacturer's instructions. Precipitated cell debris was removed by centrifugation (4500 rpm, 20 minutes) followed by filtration under vacuum through a filter plate.

Unlike the standard protocol, the DNA was then precipitated from the cleared cell lysate by adding guanidinium thiocyanate (GSCN) and triethylene glycol monomethyl ether (MTEG). 0.1-0.3 volumes of 5 M GSCN and 3 volumes 100% MTEG (v/v) solution were added to 1 volume of cleared cell lysate to precipitate the plasmid DNA. The precipitated DNA was collected and washed by filtration under vacuum through a QIAamp 96 plate, and then eluted in 125 μl warm deionized water. Without wishing to be bound by any particular theory, the inventors reasoned that in this modified step, unlike in the standard QIAGEN® protocol, the membrane in the QIAamp 96 plate simply functioned as a filter that prevented the precipitated DNA from passing though. Accordingly, any filter plate with a sufficiently small pore size could be used in this step. To confirm this, the inventors used a 0.7 μm glass fiber filter plate (Aligent®) in subsequent experiments (see Example 5) instead of the QIAGEN® QIAamp 96 plate and achieved results comparable to those described in the following paragraphs.

To further assess the quality of DNA purified according to this protocol, some of the recovered plasmid DNA was also linearized by restriction digest with HindIII-HF® (NEB, 37° C., overnight). The linearized DNA was precipitated by adding 0.1-0.3 volumes of 5 M GSCN and 3 volumes 100% MTEG (v/v) solution to 1 volume of linearized DNA. The precipitate was collected and washed (100% isopropanol) by filtration under vacuum through a QIAamp 96 plate. The DNA was eluted in 125 μl warm deionized water.

The linearized and non-linearized plasmid DNA was analyzed by agarose gel electrophoresis and the final yield was quantified with a NanoDrop2000 spectrophotometer measuring absorbance at 280 nm. The results of the gel electrophoresis for 7 plasmids purified according to this method are shown in FIG. 9B and the yields are listed in Table 3.

TABLE 3
High plasmid yields from purification including
a GSCN/MTEG DNA precipitation step

| Test plasmid | Length (kb) | Concentration (ng/μl) |
|---|---|---|
| G | 6 | 410 |
| H | 6 | 419 |
| I | 5 | 486 |
| J | 5.5 | 474 |
| K | 5.5 | 493 |
| L | 5.6 | 435 |
| M | 5.6 | 490 |

These data show that a precipitation-based DNA purification method as described above yields substantially more DNA than the standard QIAGEN® protocol. Thus, the precipitation-based purification method described in this example can be used to purify the plasmid obtained in the method described in Example 3 to serve as a template for a subsequent IVT reaction (see Example 5). Additionally, all steps of the precipitation-based purification method outlined above can be performed in 96-well plates. Therefore, the precipitation-based purification method can be readily used in a high-throughput method to purify a large number of different plasmids in parallel.

### Example 5. In Vitro Transcription Reactions

The following example describes the synthesis of mRNA by IVT of plasmids purified in accordance with the invention. These plasmids were purified according to the precipitation-based purification method using a glass fiber filter plate as described in Example 4. Each plasmid was linearized prior to the IVT reaction by restriction enzyme digestion.

In the IVT reactions, ˜2 μg of linearized plasmid (at 0.1 mg/ml) were mixed with SP6 RNA polymerase (at 0.1 mg/ml), RNase inhibitor (at 0.0003 U/μl), pyrophosphatase (0.01 μg/μl), 5 mM of each NTP, DTT (10 mM), and a reaction buffer (10× stock—250 mM Tris-HCl, pH 7.5, 20 mM spermidine, and 50 mM NaCl) in RNase free water. The reaction was incubated at 37° C. for 3 hours. The reaction was then quenched by the addition of DNase I and a DNase I buffer (10× stock—100 mM Tris-HCl, 5 mM MgCl₂ and 25 mM CaCl₂), pH 7.6) to facilitate digestion of the double-stranded DNA template in preparation for purification.

The purified mRNA product from the aforementioned in vitro transcription step was treated with portions of GTP (1.0 mM), S-adenosyl methionine, RNase inhibitor, 2′-O-methyltransferase and guanylyl transferase are mixed together with reaction buffer (10× stock—500 mM Tris-HCl (pH 8.0), 60 mM KCl, and 12.5 mM MgCl₂). The combined solution was incubated for a range of time at 37° C. for 30 to 90 minutes. Upon completion, aliquots of ATP (2.0 mM), poly(A) polymerase (100 mM stock concentration, 0.585 mM final reaction concentration) and tailing reaction buffer (provided as a 10× buffer comprising 500 mM Tris-HCl (pH 8.0), 2.5 M NaCl, and 100 mM MgCl₂) were added and the total reaction mixture was further incubated at 37° C. for a range of time from 20 to 45 minutes. Upon completion, the final reaction mixture was quenched and purified accordingly.

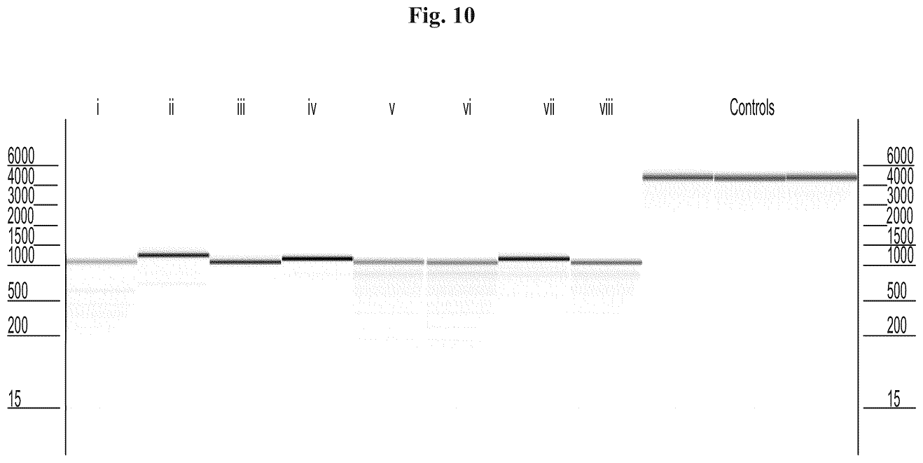

The IVT reactions were analyzed by capillary electrophoresis (FIG. 10). These results show that full-length mRNA was transcribed from the plasmids tested. These data demonstrate an IVT protocol that can be used in the screening method of the invention. In addition, the data verify that plasmids purified in accordance with the improved DNA purification protocol in the invention can be transcribed into mRNA.

### Example 6. Modified IVT Reaction Conditions for High-Throughput Assay

This example demonstrates that the composition of the in vitro transcription reaction mixture can be adjusted to provide conditions suitable for high-throughput screening of plasmids comprising nucleotide sequences of interest that vary in length and composition.

The screening methods of the invention may be employed to test a large number of nucleotide sequences encoding proteins of different lengths in parallel. Consequently, the template sequences and the resulting full-length mRNA transcripts that are screened in a single run can vary dramatically in the number of bases depending on the length of each protein in question.

The IVT reaction mixture described in Example 5 included ˜2 μg of linearized plasmid (at an input concentration of 0.1 mg/ml), SP6 RNA polymerase (at 0.1 mg/ml), RNase inhibitor (at 0.0003 U/μl), pyrophosphatase (0.01 μg/μl), 5 mM of each NTP, DTT (10 mM), and a reaction buffer (10× stock—250 mM Tris-HCl, pH 7.5, 20 mM spermidine, and 50 mM NaCl) in RNase free water. The inventors found that a total mRNA transcript yield of 120 μg per IVT reaction was desirable for downstream applications. The amount of linearized plasmid per reaction was tripled in order to improve the yield.

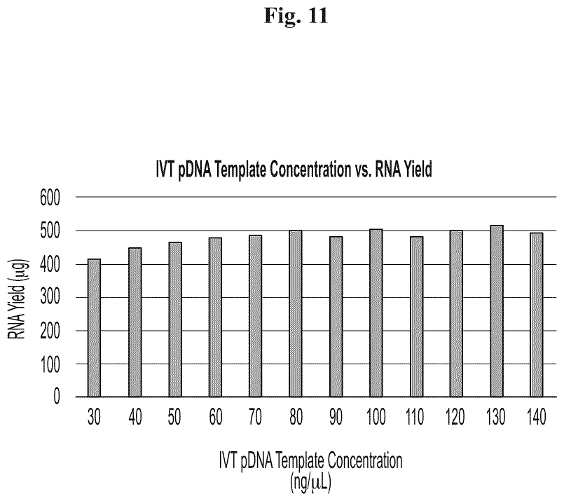

FIG. 11 shows that tripling the amount of linearized plasmid in the IVT reaction mixture to a total amount of about 6 μg (at about 0.07 mg/ml final concentration) improved the mRNA transcript yield for nucleotide sequences of interest. The SP6 RNA polymerase remained at a final concentration of about 0.1 mg/ml.

### Example 7. Post-Transcriptional Capping and Trailing

This example demonstrates that the composition of the capping/tailing reaction mixture can be adjusted to provide conditions suitable for high-throughput screening of plasmids comprising nucleotide sequences of interest that vary in length and composition.

The screening methods of the invention may be employed to test a large number of nucleotide sequences encoding proteins of different lengths in parallel. The resulting full-length mRNA transcripts that are screened in a single run can vary dramatically in the number of bases depending on the length of each protein in question. The reaction conditions for the capping and tailing of mRNA transcripts described in Example 5 do not account for the significant difference in length. It was found that these conditions can result in large variations of the poly(A) tail length (<50 nucleotides for short transcripts to >800 nucleotides for long transcripts).

To correct for the length differences of the mRNA transcripts and to achieve a more uniform poly(A) tail length, mRNA transcript size binning was tested. For molar ratio reasons, a simple rule was applied to generate the bins: no bin could have mRNA transcripts greater than 2× the size of the previous bin. This resulted in the following bins: 0 bp to 1 kb (bin 1), 1 kb to 2 kb (bin 2), 2 kb to 4 kb (bin 3), 4 kb to 8 kb (bin 4), and 8 kb+ (bin 5). The calculations for the composition of the reaction mixture for capping and tailing of mRNA transcripts were based off the largest size of the mRNA transcript in each bin (e.g., 1 kb for bin 1). Accordingly, the amount of poly(A) polymerase was halved for bin 2 relative to bin 1, and halved again for bin 3 relative to bin 2 etc.

mRNA transcripts prepared by in vitro transcription using template plasmids comprising the nucleotide sequences A-F encoding different proteins of interest were tailed applying the binning rule set out in the previous paragraph. Nucleotide sequences A-F varied in length as shown in Table 4 below:

TABLE 4

| Nucleotide sequence | mRNA transcript length (kilobases) |
|---|---|
| A | 2.4 |
| B | 1.9 |
| C | 2.1 |
| D | 2.9 |
| E | 1.1 |
| F | 0.7 |

All of the tested mRNA transcript samples were in bins 1, 2 and 3. For mRNA transcripts in bin 1, 4.8 μl of poly(A) polymerase of 1.42 mg/ml stock concentration was added to the reaction mixture for a final reaction concentration of about 166 ng/μl. The concentrations were adjusted accordingly to about 83 ng/μl and about 42 ng/μl for mRNA transcripts in bins 2 and 3 by adding half or a quarter of the poly(A) polymerase volume added to the bin 1 reaction mixture.

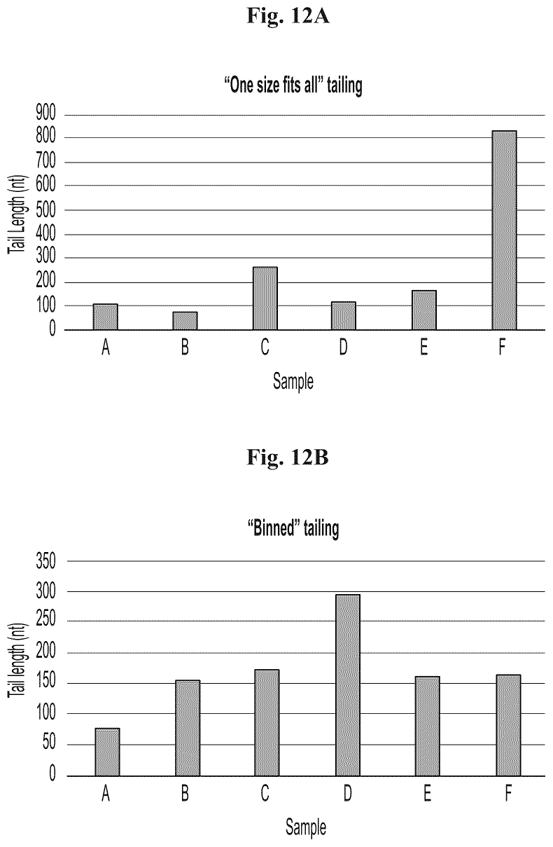

As can be seen from the comparison of FIGS. 12A and 12B, adjusting the amount of poly(A) polymerase in this manner resulted in a more uniform poly(A) tail of about 60 nucleotides to about 300 nucleotides in length for all tested mRNA transcripts. Indeed, most of the tested mRNA transcripts had a poly(A) tail of about 100-200 nucleotides in length.

### Example 8. Precipitation-Based mRNA Purification

Differences in mRNA yield in the IVT reaction can also affect subsequent purification. In fact, very high yields in the IVT reaction can create problems during subsequent purification using standard mRNA purification protocols that employ QIAGEN® spin columns or similar silica membrane-based purification systems. In particular, high mRNA transcript concentrations can result in gel formulation on the spin filter, making it difficult to wash and elute the mRNA transcripts. This negatively affects the yield of purified mRNA transcripts.

Using a precipitation-based mRNA purification protocol and a glass filter plate to capture the mRNA precipitates during washing addressed these problems. Plasmids comprising nucleotide sequences A-J encoding various proteins of interest were used as templates for in vitro transcription reactions. mRNA transcripts including nucleotide sequences A-F had the length indicated in Table 4 above. mRNA transcripts including nucleotide sequences G-J had the length shown in Table 5.

TABLE 5

| Nucleotide sequence | mRNA transcript length (kilobases) |
|---|---|
| G | 1.1 |
| H | 0.8 |
| I | 1.9 |
| J | 1.3 |

After synthesis had been completed, the template plasmid was removed by incubating the IVT reaction mixture in the presence of DNase (2.25 KU/mg plasmid) for 15 minutes at 37° C. 1 volume of the DNase-treated IVT reaction mixture was then added to 2.3 volumes of GSCN (5 M; final concentration: about 2.3 M) and 1.7 volumes of absolute isopropanol (final concentration: about 34%) to precipitate the mRNA transcripts and denature the enzymes from the IVT reaction mixture and the DNase treatment.

The resulting mRNA precipitate was then added to a 96-well glass filter plate with 1.2 μm pores (Agilent), and positive pressure was applied. The mRNA precipitate was capture on the glass filter, while the remainder of the IVT reaction mixture was removed. The glass filter was then washed with 2 ml of absolute isopropanol to purify the mRNA. The purified mRNA precipitate was readily solubilized and eluted from the filter plate with RNAse-free water to provide a high yield of purified mRNA transcripts (≥120 μg per sample).

This example demonstrates that purifying mRNA transcripts to remove enzyme reagents and other components from an in vitro transcription mixture can be achieved by precipitating the mRNA transcripts in the presence of a guanidinium salt and an alcohol. The resulting mRNA precipitate can be captured on a suitable filter medium and washed to remove any residual contaminants.

### Example 9. Automated Precipitation-Based Purification of Plasmid DNA

This example describes further improvements to the development of a precipitation-based method for the high-throughput purification of plasmid DNA. The improved method uses an automated liquid handling system and magnetic particles to (i) more effectively remove residual guanidinium salt from DNA precipitates and (ii) provide a high yield of purified plasmid DNA.

The inventors found that residual guanidinium salts left behind by precipitation-based purification processes can inhibit subsequent IVT reactions that employ an accordingly purified plasmid as a template. However, additional purification steps to remove any residual guanidinium salt from the filter plates used in Example 4 comes at the expense of a significant loss to DNA yield. Moreover, filter plates are prone to clog as the amount of input DNA is increased.

In a first step, the inventors explored the use of DNA-binding magnetic particles to avoid the disadvantages associated with using filter plates. Magnetic particles are available from a variety of suppliers. The experiments described in the present example use SeraSil-Mag 400 silica-coated beads (Cytiva). These beads bind DNA in the same way that silica filter membranes do, such as those present in QIAamp 96 plates. The use of DNA-binding magnetic particles is particularly advantageous because it is amiable to automation.

In the experiments described in this example, impure plasmid preparations of linearized plasmid DNA were provided as input to the optimized purification protocol. Other impure preparations, e.g., bacterial lysates, may also be purified with the high-throughput method described in this example. Bacterial lysates may be obtained using the protocol described in Example 4.

In an initial step, 120 μl of each impure plasmid preparation was added to separate wells of a deep-well plate (Kingfisher). To precipitate the plasmid DNA, 0.1 volumes (12 μl) of guanidine thiocyanate (GSCN), 2 volumes (240 μl) of MTEG, and 3 volumes (360 μl) of QIAGEN® PB buffer (5 M guanidine hydrochloride, 30% isopropanol) were added to each well to allow DNA precipitates to form. Then 250 μl of a solution comprising SeraSil-Mag 400 silica-coated beads (Cytiva) were added to the wells. The plate was then incubated for 5 minutes at a constant mixing speed to allow the DNA precipitates to bind to the silica-coated beads.

To purify the DNA precipitates, 900 μl of absolute isopropanol were added to all wells of a new deep-well plate. The silica-coated beads with the bound DNA precipitates were transferred to the new deep-well plate and incubated for 1 minute at a constant mixing speed (wash 1). The process was repeated with another new deep-well plate containing 900 μl of 80% (v/v) ethanol (wash 2).

To elute the purified plasmid DNA, 200 μl sterile water was added to all wells of a new deep-well plate. The washed silica-coated beads with the bound DNA precipitates were transferred to this new deep-well plate and incubated for 10 minutes at a temperature of 40° C. and constant mixing speed.

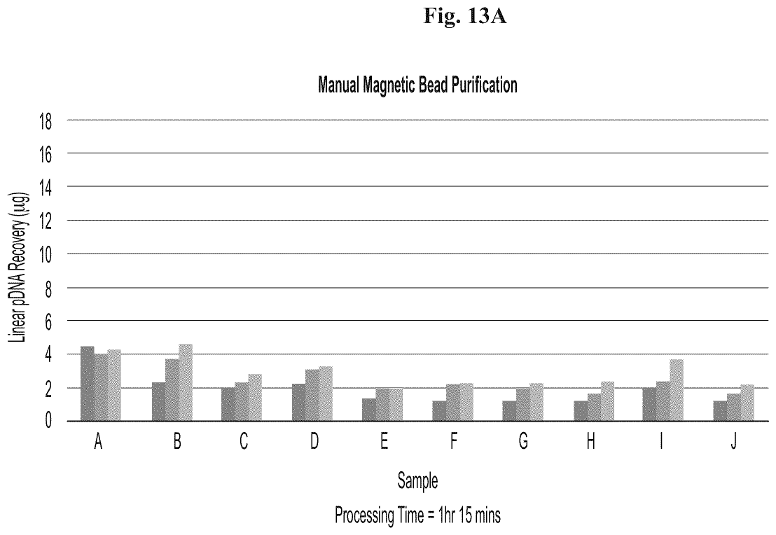

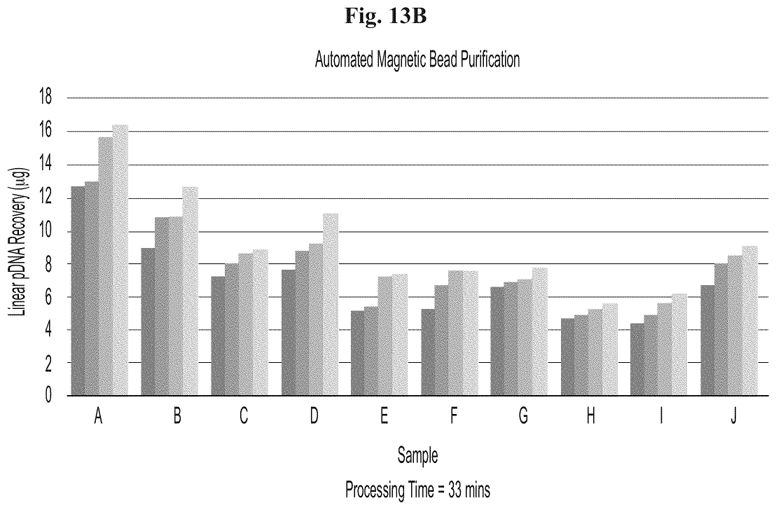

The manual transfer of liquids and beads into and between plates made the process time consuming. The overall processing time was 75 minutes. The resulting yields of covered plasmid DNA are shown in FIG. 13A. The experiment was then repeated using the same set of impure plasmid preparations. Instead of manually transferring liquids to plates, an automated liquid handling system (Kingfisher Flex Automated Magnetic Bead Platform) was used. Automation reduced the sample processing time by more than 50% to 33 minutes. Moreover, the yield of recovered plasmid DNA was dramatically increased, as can be seen in FIG. 13B.

This example demonstrates that DNA-binding magnetic particles can be used to effectively remove residual guanidinium salt from DNA precipitates. The particles can be employed with an automated liquid handling system to provide a high yield of purified plasmid DNA.

### Example 10. Binding Precipitated Plasmid to DNA-Binding Magnetic Particles

This example illustrates that increasing the guanidinium salt concentration and adding an alcohol in addition to an amphiphilic polymer during the purification of plasmid DNA improves yield when DNA-binding magnetic particles such as silica-coated beads are used.

Linearized plasmid DNA was prepared by incubating 20 μg in the presence of a suitable restriction enzyme. Once digestion had been completed, the experiment described in Example 8 was repeated using different precipitation conditions as shown in Table 6 below:

TABLE 6
Test conditions for plasmid precipitation

| | Linearized | GSCN | MTEG | Binding |
|---|---|---|---|---|
| Condition | plasmid | (5M) | (100% v/v) | solution |
| 1 | 1 volume | 0.1 volume | — | — |
| 2 | 1 volume | 0.1 volume | 1 volume | — |
| 3 | 1 volume | 0.1 volume | 2 volumes | — |
| 4 | 1 volume | 0.1 volume | 2 volumes | 3 volumes |

The binding solution contained about 30% isopropanol and 5 M guanidine hydrochloride. About 2 volumes of a solution containing the silica-coated beads was added and purification was continued as described in Example 8 above.

Each condition was tested in triplicate. The yield of purified plasmid achieved with conditions 1-3 was about 0.5 μg to 2.5 μg of total DNA. In contrast, the yield achieved with condition 4 was about 15 μg.

This example illustrates that adding a guanidinium salt at a final concentration of 2-3 M, MTEG at a final concentration of about 30-40% MTEG (v/v) and isopropanol at a final concentration of about 10-20% to an impure preparation comprising plasmid DNA results in an increased yield when DNA-binding magnetic particles such as silica-coated beads are used for high-throughput purification.

### Example 11. Vector Backbone

This example illustrates that insertion of a nucleotide sequence of interest into a vector backbone of a plasmid such that the nucleotide sequence is operationally linked to an RNA polymerase promoter can result in leaky expression of the inserted sequence in bacterial cells transformed with the plasmid. Leaky expression may be avoided by the inclusion of a bacterial terminator.

mCherry is a red fluorescent protein that can be used as an easily detectable reporter protein. A nucleotide sequence encoding the mCherry protein was assembled with a vector backbone to form a plasmid in which the nucleotide sequence was operationally linked to an SP6 RNA polymerase promoter sequence, as described in Example 3. The plasmid was used to transform electrocompetent E. coli cells (HST08 and DH10B). With both cell types, mCherry expression was observed when transformed E. coli cells were cultured in 2 ml Terrific Broth. This suggests that an endogenous bacterial RNA polymerase was transcribing the inserted nucleotide sequence using the SP6 RNA polymerase promoter sequence.

Leaky expression can be detrimental to bacterial host cells and result in the selection of mutations that lead to reduced expression or plasmid copy numbers or result in the inactivation of the protein encoded by the nucleotide sequence of interest. Faithful propagation of a plasmid comprising a nucleotide sequence of interest is imperative to ensure a continued supply of the plasmid as a template for in vitro transcription, in particular for the event that the nucleotide sequence of interest is selected for the manufacturing of a therapeutic mRNA.

To avoid leaky expression in bacterial cells, the vector backbone was further optimized. In particular, two bacterial terminators were tested for inclusion in the vector backbone. One was the ropC terminator from E. coli strain JME66 (SEQ ID NO: 28), the other was the bla terminator from Staphylococcus aureus strain 16405 (SEQ ID NO: 29).

1. A screening method comprising:
a. providing a plurality of nucleotide sequences encoding a protein generated by a sequence optimization algorithm;
b. for each nucleotide sequence, providing two or more DNA fragments with a first set of homologous ends and a second set of homologous ends, wherein said two or more DNA fragments, when assembled via the first set of homologous ends, yield an insert with the second set of homologous ends and comprising the nucleotide sequence;
c. providing two or more vector fragments with the second set of homologous ends and a third set of homologous ends, wherein said two or more vector fragments, when assembled via the third set of homologous ends, yield a vector backbone with the second set of homologous ends;
d. for each nucleotide sequence, assembling the two or more DNA fragments and the two or more vector fragments via the first, second and third sets of homologous ends, wherein assembly of the insert and the vector backbone via the second set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

2. A screening method comprising:
a. providing a plurality of nucleotide sequences encoding a protein generated by a sequence optimization algorithm;
b. for each nucleotide sequence, providing two or more DNA fragments with a first set of homologous ends and a second set of homologous ends, wherein said two or more DNA fragments, when assembled via the first set of homologous ends, yield an insert with the second set of homologous ends and comprising the nucleotide sequence;
c. providing a vector backbone with the second set of homologous ends;
d. for each nucleotide sequence, assembling the two or more DNA fragments and the vector backbone via the first and second sets of homologous ends, wherein assembly of the insert and the vector backbone via the second set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

3. A screening method comprising:
a. providing a plurality of nucleotide sequences encoding a protein generated by a sequence optimization algorithm;
b. for each nucleotide sequence, providing an insert with a first set of homologous ends and comprising the nucleotide sequence;
c. providing two or more vector fragments with the first set of homologous ends and a second set of homologous ends, wherein said two or more vector fragments, when assembled via the second set of homologous ends, yield a vector backbone with the first set of homologous ends;
d. for each nucleotide sequence, assembling the insert and the two or more vector fragments via the first and second sets of homologous ends, wherein assembly of the insert and the vector backbone via the first set of homologous ends yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

4. A screening method comprising:
a. providing a plurality of nucleotide sequences encoding a protein generated by a sequence optimization algorithm;
b. for each nucleotide sequence, providing an insert comprising the nucleotide sequence and a set of homologous ends;
c. providing a vector backbone comprising the set of homologous ends;
d. for each nucleotide sequence, assembling the insert and the vector backbone via the set of homologous ends, wherein the assembly yields a plasmid comprising the nucleotide sequence flanked by a 5′ untranslated region (5′ UTR) and a 3′ untranslated region (3′ UTR) and operationally linked to an RNA polymerase promoter, wherein the 5′ UTR, the 3′ UTR and the RNA polymerase promoter are either part of the insert or the vector backbone;
e. adding each plasmid to an in vitro transcription reaction mixture to transcribe the nucleotide sequence into an mRNA transcript; and
f. selecting nucleotide sequences that generate a full-length mRNA transcript.

5. The method of any one of the preceding claims, further comprising the following steps:
g. for each nucleotide sequence selected in step (f), transfecting a cell with the full-length mRNA transcript;
h. for each cell transfected in step (g), determining the amount of the encoded protein expressed from the full-length mRNA transcript; and
i. selecting the nucleotide sequence, whose full-length mRNA transcript yields the largest amount of the encoded protein.

6. The method of any one of the preceding claims, wherein the sequence optimization algorithm comprises the steps of:
(i) receiving an amino acid sequence encoding the protein;
(ii) receiving a first codon usage table, wherein the first codon usage table comprises a list of amino acids, wherein each amino acid in the table is associated with at least one codon and each codon is associated with a usage frequency;
(iii) removing from the codon usage table any codons associated with a usage frequency which is less than a threshold frequency;
(iv) generating a normalized codon usage table by normalizing the usage frequencies of the codons not removed in step (iii);
(v) generating a nucleotide sequence encoding the amino acid sequence by selecting a codon for each amino acid in the amino acid sequence based on the usage frequency of the one or more codons associated with the amino acid in the normalized codon usage table; and
(vi) repeating step (v) to generate the plurality of nucleotide sequences.

7. The method of claim 6, wherein the sequence optimization algorithm further comprises the steps of:
(vii) determining the codon adaptation index of each of the nucleotide sequences, wherein the codon adaptation index of a sequence is a measure of codon usage bias and can be a value of 0 to 1;
(viii) removing any nucleotide sequence if its codon adaptation index is less than or equal to a predetermined codon adaptation index threshold.

8. The method of claim 7, wherein the codon adaptation index threshold is 0.7, or 0.75, or 0.85, or 0.9, or, in particular, 0.8.

9. The method of any one of the preceding claims, wherein sequence optimization algorithm comprises the steps of:
i. determining whether any one of the nucleotide sequences contains a termination signal; and
ii. removing any nucleotide sequence if the nucleotide sequence contains one or more termination signals.

10. The method of claim 9, wherein the one or more termination signals has/have the following nucleic acid sequence:
5′-X₁ATCTX₂TX₃-3′,
wherein X₁, X₂ and X₃ are independently selected from A, C, T or G.

11. The method of claim 10, wherein the one or more termination signals has/have one or more of the following nucleotide sequences:
TATCTGTT;
and/or
TTTTTT;
and/or
AAGCTT;
and/or
GAAGAGC;
and/or
TCTAGA.

12. The method of any one of the preceding claims, wherein each of the nucleotide sequences provided in step (a) is processed by a fragmentation algorithm, wherein the fragmentation algorithm divides each nucleotide sequence into two or more nucleic acid fragments and adds the homologous ends required for the assembling of the plasmid performed in step (d).

13. The method of any one of the preceding claims, wherein each of the two or more DNA fragments or the insert, as applicable, are provided by a chemical synthesis process.

14. The method of claim 13, wherein each of the two or more DNA fragments or the insert is about or at least 1000 base pairs long.

15. The method of claim 14, wherein each of the two or more DNA fragments or the insert is 1000 base pairs to 4000 base pairs long.

16. The method of claim 14, wherein each of the two or more DNA fragments or the insert is 1000 base pairs to 7000 base pairs long.

17. The method of claim 14, wherein each of the two or more DNA fragments or the insert is 1000 base pairs to 20,000 base pairs long.

18. The method of any one of claims 13-17, wherein the chemical synthesis process has a median error rate of less than or equal to 1 error per 5000 base pairs.

19. The method of claim 18, wherein the chemical synthesis process has a median error rate of less than or equal to 1 error per 10,000 base pairs.

20. The method of claim 18, wherein the chemical synthesis process has a median error rate of less than or equal to 1 error per 50,000 base pairs.

21. The method of any one of the preceding claims, wherein the homologous ends are 15 base pairs to 30 base pairs long.

22. The method of claim 1 or claim 3, wherein step (c) is performed by means of a DNA polymerase and a plurality of primer pairs encoding the second and third sets of homologous ends or the first and second sets of homologous ends, as applicable.

23. The method of any one of the preceding claims, wherein step (d) is performed in the presence of a DNA polymerase and/or an exonuclease, and optionally a ligase.

24. The method of claim 23, wherein step (d) is performed in the presence of a 5′ exonuclease, a DNA polymerase and a ligase.

25. The method of any one of the preceding claims, wherein the vector backbone comprises a negative selection marker gene and/or a positive selection marker gene.

26. The method of any one of the preceding claims, wherein the vector backbone comprises an origin of replication having the nucleotide sequence of SEQ ID NO: 2.

27. The method of any one of the preceding claims, wherein the plasmid comprises a bacterial terminator, wherein the bacterial terminator is located in the vector backbone upstream of the RNA polymerase promoter.

28. The method of claim 27, wherein the bacterial terminator is an Escherichia coli ropC terminator.

29. The method of claim 27, wherein the bacterial terminator is a Staphylococcus aureus bla terminator.

30. The method of any one of the preceding claims, wherein the vector backbone comprises two or more termination signals arranged sequentially and positioned at the 3′ end of the 3′UTR in the plasmid.

31. The method of claim 30, wherein the plasmid comprises three termination signals.

32. The method of claim 30 or claim 31, wherein each termination signal comprises the following nucleic acid sequence:
5′-X₁ATCTX₂TX₃-3′,
wherein X₁, X₂ and X₃ are independently selected from A, C, T or G.

33. The method of claim 32, wherein each termination signal comprises the nucleic acid sequence 5′-X₁ATCTGTT-3′.

34. The method of claim 32 or claim 33, wherein X₁ is T.

35. The method of claim 32 or claim 33, wherein X₁ is C.

36. The method of any one of claims 32-35, wherein the termination signal is selected from

| (SEQ ID NO: 3) | |
|---|---|
| | 5′-TTTTATCTGTTTTTTT-3′, |
| (SEQ ID NO: 4), | |
| | 5′-TTTTATCTGTTTTTTTTT-3′ |
| (SEQ ID NO: 5) | |
| | 5′-CGTTTTATCTGTTTTTTT-3′, |
| (SEQ ID NO: 6) | |
| | 5′-CGTTCCATCTGTTTTTTT-3′, |
| (SEQ ID NO: 7) | |
| | 5′-CGTTTTATCTGTTTGTTT-3′, |
| | or |
| (SEQ ID NO: 8) | |
| | 5′-CGTTTTATCTGTTGTTTT-3′. |

37. The method of any one of claims 30-36, wherein each termination signal is separated by 10 base pairs or fewer, e.g., separated by 5-10 base pairs.

38. The method of any one of claims 1-29, wherein the plasmid is linearized before step (e).

39. The method of any one of the preceding claims, wherein the RNA polymerase promoter is a SP6 polymerase promoter.

40. The method of any one of the preceding claims, further comprising one or more purification steps prior to performing step (e).

41. The method of claim 40, wherein the one or more purification steps comprises purifying the plasmid to remove one or more enzyme(s) used for assembly in step (d).

42. The method of claim 40, wherein the one or more purification steps comprises extracting the plasmid from Escherichia coli cells.

43. The method of any one of claims 40-42, wherein the purification comprises precipitating plasmid by adding (i) a chaotropic salt and (ii) an alcohol and/or an amphiphilic polymer.

44. The method of claim 43, wherein the chaotropic salt is at a final concentration of 0.1-4 M.

45. The method of claim 44, wherein the chaotropic salt is at a final concentration of about 125 mM, about 250 mM, about 375 mM, about 500 mM, about 625 mM, about 750 mM, about 1.3 M, about 1.9 M or about 2.5 M.

46. The method of any one of claims 43-45, wherein the chaotropic salt is guanidinium salt, optionally guanidinium thiocyanate (GSCN).

47. The method of any one of claims 43-46, wherein the amphiphilic polymer is selected from pluronics, polyvinyl pyrrolidone, polyvinyl alcohol, polyethylene glycol (PEG), triethylene glycol monomethyl ether (MTEG), or combinations thereof.

48. The method of claim 47, wherein the amphiphilic polymer is MTEG.

49. The method of any one of claims 43-46, wherein the alcohol is isopropanol or ethanol.

50. The method of any one of the preceding claims, wherein the in vitro transcription reaction mixture in step (e) comprises the plasmid at a concentration of 0.05 mg/ml or greater, e.g., at a concentration of about 0.07 mg/ml.

51. The method of any one of the preceding claims, wherein the in vitro transcription reaction mixture comprises an RNA polymerase at a concentration of 0.05 mg/ml or greater, e.g., at a concentration of about 1 mg/ml.

52. The method of any one of the preceding claims, wherein steps (b)-(f) are performed in 96-well plates.

53. The method of any one of the preceding claims, wherein steps (g)-(i) are performed in 96-well plates.

54. The method of any one of the preceding claims, wherein the cell transfected in step (h) is a mammalian cell.

55. The method of claim 54, wherein the mammalian cell is a human cell.

56. A high-throughput method for purifying a plurality of DNA constructs, wherein the method comprises performing for each DNA construct the following steps in parallel:
a. providing an impure preparation comprising the DNA construct in a first receptacle;
b. adding (i) a chaotropic salt, (ii) an alcohol and/or an amphiphilic polymer, and optionally (iii) a buffered solution to the impure preparation under conditions that result in the formation of a precipitate comprising the DNA construct;
c. adding a DNA-binding magnetic particle to bind the precipitate formed in step (b) to the magnetic particle;
d. transferring the magnetic particle with the bound precipitate from the first receptacle to a second receptacle comprising a first wash solution;
e. optionally transferring the magnetic particle with the bound precipitate from the second receptacle to a third receptacle comprising a second wash solution;
f. transferring the magnetic particle with the bound precipitate from the wash solution to a fourth receptacle comprising an elution medium; and
g. solubilizing the precipitate in the elution medium to release the purified DNA construct.

57. The high-throughput method of claim 56, wherein each of the first, second, third and fourth receptacles is a well in a first, second, third and fourth multi-well plate, respectively.

58. The high-throughput method of claim 57, wherein each multi-well plate is a 96-well plate.

59. The high-throughput method of any one of claims 56-58, wherein each step is performed by an automated liquid handling system.

60. The high-throughput method of any one of claims 56-59, wherein the magnetic particle is a silica-coated bead with a metallic core.

61. The high-throughput method of claim 60, wherein the metallic core comprises iron, nickel or cobalt.

62. The high-throughput method of any one of claims 56-61, wherein the chaotropic salt is at a final concentration of 0.1-4 M to form the precipitate in step (b).

63. The high-throughput method of claim 62, wherein the chaotropic salt is at a final concentration of 1.5 M-2.7 M.

64. The high-throughput method of any one of claims 56-63, wherein the chaotropic salt is a guanidinium salt, optionally guanidinium thiocyanate (GSCN).

65. The high-throughput method of any one of claims 56-64, wherein the amphiphilic polymer is selected from pluronics, polyvinyl pyrrolidone, polyvinyl alcohol, polyethylene glycol (PEG), triethylene glycol monomethyl ether (MTEG), or combinations thereof.

66. The high-throughput method of claim 65, wherein the amphiphilic polymer is MTEG.

67. The high-throughput method of claim 65 or claim 66, wherein the amphiphilic polymer is present at about 30% (v/v) to about 70% (v/v) final concentration to form the precipitate in step (a).

68. The high-throughput method of any one of claims 56-64, wherein the alcohol is isopropanol or ethanol.

69. The high-throughput method of claim 68, wherein ethanol is present at about 10% (v/v) to about 70% (v/v) final concentration to form the precipitate in step (a).

70. The high-throughput method of any one of claims 56-69, wherein the buffered solution has a pH of about 5 to about 6.

71. The high-throughput method of any one of claims 56-70, wherein the buffered solution comprises potassium acetate.

72. The high-throughput method of claim 71, wherein an amount of the buffered solution is provided in step (b) to obtain a final potassium acetate concentration of about 1 M to about 2 M.

73. The high-throughput method of any one of claims 56-72, wherein the first wash solution is 100% isopropanol or 80% (v/v) ethanol.

74. The high-throughput method of any one of claims 56-73, wherein the second wash solution is 100% isopropanol or 80% (v/v) ethanol.

75. The high-throughput method of any one of claims 56-74, wherein the first wash solution is 100% isopropanol and the second wash solution is 80% (v/v) ethanol.

76. The high-throughput method of any one of claims 56-74, wherein the first wash solution and the second wash solution are 80% (v/v) ethanol.

77. The high-throughput method of any one of claims 56-74, wherein the first wash solution and the second wash solution are 100% isopropanol.

78. The high-throughput method of any one of claims 56-77, wherein the elution medium is sterile water.

79. The high-throughput method of any one of claims 56-78, wherein the elution medium is heated to a temperature of 30° C. to 50° C. to enhance solubilization of the precipitate.

80. The high-throughput method of any one of claims 56-79, wherein the impure preparation is a cell lysate.

81. The high-throughput method of claim 80, wherein the cell is a bacterial cell.

82. The high-throughput method of claim 80 or claim 81, wherein the lysate is an alkaline solution.

83. The high-throughput method of any one of claims 80-82, wherein the lysate comprises a detergent.

84. The high-throughput method of any one of claims 56-83, wherein the method further comprises determining the concentration of the purified DNA construct obtained in step (g).

85. The high-throughput method of any one of claims 56-84, wherein the method further comprises lyophilizing the purified DNA construct obtained in step (g).

86. The high-throughput method of any one of claims 56-85, wherein each DNA construct in the plurality of DNA constructs is a plasmid.

87. The high-throughput method of claim 86, wherein each plasmid comprises a nucleotide sequence, said nucleotide sequence being flanked by a 5′ untranslated region (5′ UTR) and a 3′ untranslated region (3′ UTR) and operationally linked to an RNA polymerase promoter.

88. The high-throughput method of claim 87, wherein each preparation of purified plasmid obtained in step (g) comprises the chaotropic salt at a concentration that does not interfere with in vitro transcription of the nucleotide sequence.

89. The high-throughput method of claim 87 or claim 88, wherein the nucleotide sequence was generated by a codon optimization algorithm.
